(19) United States
(12) Patent Application Publication
(10) Pub. No.: US 2025/0243233 A1
(43) Pub. Date: Jul. 31, 2025

(54) BIOBASED SURFACTANTS

(71) Applicant: ECOLE POLYTECHNIQUE FEDERALE DE LAUSANNE (EPFL), Lausanne (CH)

(72) Inventors: Anastasiia KOMAROVA, Chavannes-Renens (CH); Songlan SUN, Ecublens (CH); Stefania BERTELLA, Espoo (FI); Jeremy LUTERBACHER, Chavannes près Renens (CH)

(21) Appl. No.: 18/855,838

(22) PCT Filed: Apr. 11, 2023
(86) PCT No.: PCT/EP2023/059392

§ 371 Date: Oct. 10, 2024

(30) Foreign Application Priority Data
Apr. 11, 2022 (EP) 22167726.3
Jul. 28, 2022 (EP) 22187550.3

(51) Int. Cl. C07H 9/04; A61K 8/49; A61K 8/60; A61K 47/22; A61K 47/26; A61Q 19/00; C07D 307/20; C09K 23/32

(52) U.S. Cl. CPC C07H 9/04; A61K 8/4973; A61K 8/602; A61K 47/22; A61K 47/26; A61Q 19/00; C07D 307/20; C09K 23/32; A61K 2800/10

(57) ABSTRACT

Compound of the general formula (Ia), (Ib) and (Ic) R₅₀ and R₆₀ are different form each other and are selected from the group consisting of —R₇₀, —ZR₇₀, —Z—OH, —Z—NH₂, —Z—SH, —Z—OC(O)R₇₀, —OC(O)R₇₀, —COOH and its corresponding salts, —C(O)NH₂, —C(O)NH—R₇₀, —C(O)N—(R₇₀)₂, —COOR₇₀, —Z—COOH and its corresponding salts, —Z—C(O)NH—R₇₀, —Z—C(O)NH₂, —Z—C(O)N—(R₇₀)₂, —Z—COOR₇₀, —CH(COOH)₂ and its corresponding salts, —CH(COOR₇₀)₂, and —Z—SO₃— wherein R₇₀ is selected from the group consisting of a linear or branched C₁ to C₂₀ alkyl, (C₁ to C₁₀)-alkyloxy-(C₁ to C₁₀)-alkyl, C₂ to C₁₀ alkenyl, C₆ to C₁₂ aryl, C₃ to C₁₀ cycloalkyl, cycloalkylalkyl and cycloalkylalkenyl, wherein Z is a linear or branched C₁ to C₁₀ alkyl, linear or branched C₃ to C₁₀ cycloalkyl, a linear or branched C₆ to C₁₀ aryl or a (C₁ to C₁₀)-alkyloxy-(C₁ to C₁₀)-alkyl, cycloalkylalkyl and cycloalkylalkenyl.

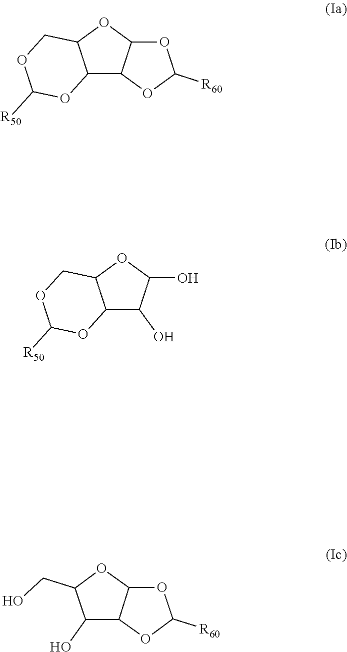

The present invention relates to new compounds and their use as biobased surfactants.

Surfactants are a class of chemicals used in a wide range of applications and fields such as the detergent, medical, pharmaceutical, food and paint industries. With the advent of the COVID-19 global pandemic, the demand of surfactants has risen, since these compounds contained in soaps and sanitizing compositions have the ability of disrupting the lipidic membrane of the SARS-CoV-2 virus making said virus inefficient.

Given the huge market and big demand for surfactants, it is important that their production does not rely only on fossil-based sources, but more and more it is fundamental that they are synthesized and sourced from renewable feedstocks that don't impact as much on the second current global challenge, climate change.

Moreover, as the lifetime of surfactant is short, given that they are generally washed away and tend to end up in the open, their structure had degradability products have to be compatible with the environment they end up in, so that they neither accumulate nor cause harm to the diverse species and ecosystems.

Bio-based surfactants (Sophorolipids, Rhamnolipids) are commercially available. Sophorolipids are commercialised by Ecover, Saraya, Intobio, Evonik™ and Allied Carbon Solutions™. All of them have a critical micelle concentration CMC 7-10-fold less than CMC of SDS but need to be produced with yeasts (average fermentations times: 7 days) and with fatty acids of tropical plant origin, that still pose an environmental pressure, due to deforestation issues.

US2021353517A1 discloses a process for producing a bio-based surfactant comprising an alkyl disulphate salt comprises the steps of methanolysis of medium chain length polyhydroxyalkanoic acid (mcl-PHA) to provide hydroxy fatty acid methyl ester monomers (HFAME's), reduction of the HFAME's to provide 1,3 alkyl diols, sulphation of the 1,3 alkyl diols to provide 1,3 alkyl disulphates, and neutralisation of the alkyl disulphates to provide a bio-based surfactant comprising 1,3 alkyl disulphate salt.

Particularly, carbohydrates (or their derivatives)-based surfactants have received a lot of attention and development for the variety of possible chemical reactions that are possible on the hydroxyl group of their core structure. Typical carbohydrate-based molecules or derivatives that can be chemically reacted to form surfactants are xylose, glucose, sorbitol, sorbitan, arabinose, isosorbide, and uronic acid. Amongst the possible reaction that have been studied in making sugar-based surfactants the most common are the esterification of carbohydrate hydroxyl group with long chain acids, such as the case of the commercial Span and Tween derived from sorbitol. Other important reactions of carbohydrates to produce surfactants are etherification to form simple ether or reactions with long-chain aldehydes to form stable acetals. Finally, in the case of uronic acid, amide-based surfactants can be synthesized. Some of these reactions, although successful (such as the case of commercial esters based on sorbitan), are based on sugar or derivatives that undergo several synthetic steps of reduction, isomerization or dehydration of more abundant sugars before becoming surfactants, increasing their environmental and energetical impact with each synthetic step.

The problem of the present invention is therefore to provide bio-based surfactant which can be synthesized in a few steps directly from renewable sources.

The problem is solved by the compounds according to claim 1. Further preferred embodiments are subject of the dependent claims.

As explained in detail below, compounds of formula Ia, Ib, Ic, II and III can be obtained based on aldehyde assisted biomass fractionation and acetal functionalization from carbohydrate-based molecules. The compounds of the present invention are biodegradable and have no negative impact on human and animal health. In addition, they have no or only a very limited negative influence on fauna, flora, and ecosystems since they are derived from renewable resources. Furthermore, the synthesis of the compounds according to the present invention is simple which allows a large-scale bio-based surfactant production.

The present invention relates to a compound of formula (Ia), (Ib) or (Ic)

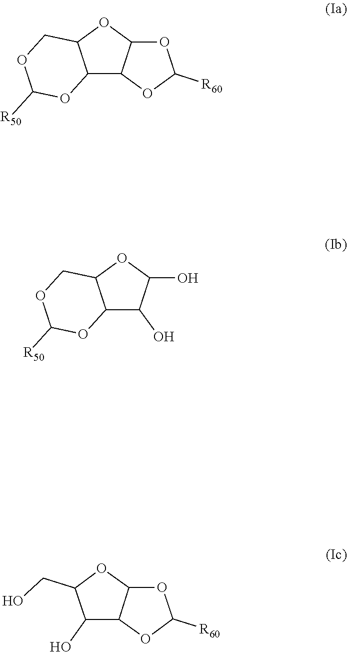

wherein R₅₀ and R₇₀ are different form each other and are selected from the group consisting of —R₇₀, —ZR₇₀, —Z—OH, —Z—NH₂, —Z—SH, —Z—OC(O)R₇₀, —CC(O)R₁₀, —COOH and its corresponding salts, —C(O) NH₂, —C(O)NH—R₇₀, —C(O)N—(R₇₀)₂, —COOR₇₀, —Z—COOH and its corresponding salts, —Z—C(O)NH—R₇₀, —Z—C(O)NH₂, —Z—C(O)N—(R₇₀)₂, —Z—COOR₇₀, —CH(COOH)₂ and its corresponding salts, —CH(COOR₇₀)₂, and —Z—SO₃—
wherein R₇₀ is selected from the group consisting of a linear or branched C₁ to C₂₀ alkyl, (C₁ to C₁₀)-alkyloxy-(C₁ to C₁₀)-alkyl, C₂ to C₁₅ alkenyl, C₆ to C₁₂ aryl, C₃ to C₁₀ cycloalkyl, cycloalkylalkyl and cycloalkylalkenyl,
wherein Z is a linear or branched C₁ to C₁₀ alkyl, linear or branched C₃ to C₁₀ cycloalkyl, a linear or branched C₆ to C₁₀ aryl or a (C₁ to C₁₀)-alkyloxy-(C₁ to C₁₀)-alkyl, cycloalkylalkyl and cycloalkylalkenyl.

The term “linear or branched C₁ to C₂₀ alkyl” as a residue refers to a linear or branched hydrocarbon chain containing 1 to 20 of carbon atoms. Examples of said term as used herein include methyl, ethyl, n-propyl, isopropyl, n-butyl, isobutyl, sec-butyl, tert-butyl, n-pentyl, 3-methylbutyl, sec-pentyl, and 2-methylbutan-2-yl.

The term “a (C₁ to C₁₀)-alkyloxy-(C₁ to C₁₀)-alkyl” as a residue refers to a residue —(C₁-C₁₀)—O—(C₁-C₁₀)—, i.e., a linear or branched hydrocarbon chain containing preferably 1 to 20 of carbon atoms, of which at least two are singularly bonded to oxygen. Examples of said term as used herein include 2-ethylethoxy, 2-ethylpropoxy, 2-ethylpropoxy, 1-ethylbutoxy, 3-ethylbutoxy, and 2-ethylpropoxy.

The term “linear or branched C₂ to C₂₀ alkenyl” as a residue refers to a linear or branched hydrocarbon chain containing 2 to 20 of carbon atoms which contains at least one double bond. The double bond can be in any position of the linear or branched hydrocarbon chain. In branched hydrocarbon chains the double bond is typically located between two adjacent carbon atoms in the longest continuous carbon chain, which may include one or more branches. Preferably, the position of the double bond is directly adjacent to the ring system, i.e., in alpha-position to the ring system. One possible example is —CH═CH—(CH₂)₈—CH₃.

The term “C₆ to C₁₂ aryl” refers to an aromatic carbon ring system having any suitable number of ring atoms and any suitable number of rings. Aryl groups can include any suitable number of carbon ring atoms, such as, 6, 7, 8, 9, 10, 11 or 12 ring atoms, as well as from 6 to 10 or 6 to 12 ring members. Aryl groups can be monocyclic, fused to form bicyclic or tricyclic groups, or linked by a bond to form a biaryl group. Representative aryl groups include phenyl, naphthyl and biphenyl. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

The term “cycloalkyl” means a non-aromatic monocyclic ring system comprising 3 to 10 carbon atoms, preferably 5 to 10 carbon atoms. Preferred cycloalkyl rings contain about 5 to about 7 ring atoms. Non-limiting examples of suitable monocyclic cycloalkyls include cyclopentyl, cyclohexyl, cycloheptyl and the like.

The term “cycloalkylalkyl” means a residue —RₐR_{b} where Rₐ is an alkylene group having 1 to 8 carbon atoms and R_{b} is cycloalkyl group as defined above.

The term “cycloalkylalkenyl” means a residue —Rₐ′R_{b} where Rₐ′ is an alkenylene group having 2 to 8 carbon atoms and R_{b} is cycloalkyl group as defined above.

The term “or its corresponding salt” means the carboxylate salt which is formed by the reaction of a carboxylic acid with a base, i.e sodium carboxylate.

The compound of the present invention is preferably used as a non-ionic surfactant, which in general comprises an acetal-stabilized sugar core and residual groups R (for example R₁₁, or R₃₁) and has the same properties as polyoxyethylene-based surfactants and sugar-derived polyols.

Polyoxyethylene-based surfactants exhibit inverse solubility characteristics and may precipitate with temperature increase. At low temperatures, the chains of the polyoxyethylene-based surfactant are able to interact with water molecules, keeping the surfactant molecules soluble in the aqueous solution. However, as the temperature increases, the thermal motion of water molecules becomes more energetic, which reduces the strength of the hydrogen bonding interactions between the POE chains and water molecules.

Sugar-derived polyols such as compounds having a sugar core functionalized with pH-responsive functional group, e.g. a carboxylic acid (—COOH), or an amine (—NH₂), are very pH-responsive. Their solubility strongly depends on the pH and some of the linkages are not very stable either under very acidic or under very basic condition Therefore, they can be easily degraded after use without burdening the environment. On the other hand, with the exchange of the functional group a sugar-derived polyol can be adapted from working in a lower pH range to work in the adapted form in a higher pH range.

These combined properties allow for a complex, multi responsive behaviour of the compounds according to the present invention, when temperature and pH of the solution are changed. Resulting potential applications comprise the use as a thermo-responsive and chelating amino-acid modified surfactants, temperature-responsive functional surfactants, tethered lipid membranes with a variable hydrophilic cushion or hydrophilic and temperature-responsive drug carriers. Further these compounds can be used as surfactants in household and industrial cleaning agents.

Due to the variety of residual groups of the claimed compound, different kinds of effects can be expected.

Compounds containing ester groups are more sensitive towards hydrolyses, especially in alkaline conditions. This means that esters are preferably used as emulsifiers and stabilizers under neutral conditions in personal care, food and pharmaceutical applications.

Compounds containing amide groups are relatively more stable than compounds containing an ester group and tend to be hydrolysed only in harsh conditions such as a strong acid (e.g. H₂SO₄) in combination with heat or a strong base (e.g. NaOH) in combination with heat.

Compounds containing alkyl ethers groups are relatively stable in alkaline surroundings but under acidic catalysis they can turn into furans, and subsequently humins (sugar degradation products). They can be used in cleaning under mild conditions, such as in personal care, dish-washing liquids, etc., but also for applications where alkaline conditions are required. It is expected that compounds with alkyl ethers groups are resistant to hard water conditions (between 120-180 mg CaCO₃/L).

Compounds containing carboxylic acid groups opens up a multitude of possible applications due to their pH-responsive character. The degree of ionization of carboxylic acid residue depends on the pH of the solution.

Moreover, the high reactivity of the carboxylic acid residues enables the preparation of surfactants tailored to specific purposes. Due to the simultaneous presence of the ether units and the carboxylic acid residues, clear solutions of surfactants with long alkyl chains (C₁₂-C₁₈) can be obtained in alkaline, as well as acidic solutions which are remarkably stable in hard water and high salinity conditions. In combination with their low toxicity, stability to hydrolysis, oxidizing agents, high temperatures, they will find application in different fields, most notably as detergents and in home and body-care products, for enhanced oil recovery and as additives in the textile and metal processing industry.

One preferred embodiment of the present invention relates to the compounds (Ib) or (Ic), wherein R₅₀ or R₆₀ is —R₇₀ and wherein —R₇₀ is a linear C₇ to C₁₉ alkyl, preferably a linear C₉ to C₁₇ alkyl and more preferably a linear C₁₁ alkyl.

Linear alkyl chains and in particular dodecanal (C₁₂ aldehyde) can be used as starting material for the production of compounds (Ib) or (Ic) that have a linear alkyl chain. These linear alkyl chains can be synthesized sustainably in the industry using plant oils such as coconut oil, palm oil, or castor oil via a process that involves hydrolysis, reduction, and selective oxidation. Compared to alkenyls, alkoxys, and aryls, linear alky chains and in particular dodecanal are relatively low-cost raw materials.

In acetalization reactions, the strong electrophilicity of the carbonyl group in alkyl aldehydes makes it possible to produce high-stability products with high yields. Compounds (Ib) and (Ic) with a C₁₁ alkyl chain have a desirable balance of hydrophilicity and hydrophobicity due to their C₁₁ alkyl chains. This unique property makes them valuable in a variety of applications where a balance between water solubility and oil solubility is required.

One preferred embodiment of the present invention relates to the compound (Ib) wherein R₅₀ is —R₇₀ and wherein —R₇₀ is a linear C₇ to C₁₉ alkyl, preferably a linear C₉ to C₁₇ alkyl and more preferably a linear C₁₁ alkyl.

One preferred embodiment of the present invention relates to the compound (Ic), wherein R₆₀ is —R₇₀ and wherein —R₇₀ is a linear C₇ to C₁₉ alkyl, preferably a linear C₉ to C₁₇ alkyl and more preferably a linear C₁₁ alkyl.

One preferred embodiment of the present invention relates to the compound (Ib) wherein R₅₀ is —R₇₀ and wherein —R₇₀ is a linear C₂ to C₁₅ alkenyl, preferably a linear C₉ to C₁₃ alkenyl and more preferably a linear C₁₁ alkenyl.

One preferred embodiment of the present invention relates to the compound (Ib) wherein R₅₀ is —R₇₀ and wherein —R₇₀ is a linear C₂ to C₁₅ alkenyl, preferably a linear C₉ to C₁₃ alkenyl and more preferably a linear C₁₁ alkenyl and wherein the double bond is preferably in alpha position.

The present invention further relates to a compound of the general formula (V),

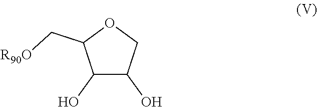

wherein R₉₀ is selected from the group consisting of a linear or branched C₁ to C₂₀ alkyl, (C₁ to C₁₀)-alkyloxy-(C₁ to C₁₀)-alkyl, C₂ to C₁₀ alkenyl, C₆ to C₁₂ aryl, C₃ to C₁₀ cycloalkyl, cycloalkylalkyl and cycloalkylalkenyl.

One embodiment of the present invention relates to compound (V) wherein R₉₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃, preferably selected from the group consisting of —(CH₂)₁₁CH₃.

One embodiment of the present invention relates to compound (Va),

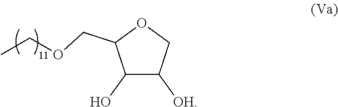

Compound (V) can be synthesized by performing Palladium-catalyzed hydrogenolysis on starting material compound (Ia) with symmetric chain group. This synthesis is easy to perform and reduces the cost. As described above the compound (Ia) with C₁₁ alkyl chains has a desirable balance of hydrophilicity and hydrophobicity. Therefore, compound (Ia) with a C₁₁ alkyl chains is the preferred starting material, which results in compound (Va) with a C₁₂ alkyl residual group.

The compound of the general formula (Ia), (Ib), (Ic) and (V) of the present invention can be used as surfactants, preferably as emulsifiers, foam stabilizers, wetting agents, emollients in cosmetic products, surfactants in food products, as anti-spattering agents for frying or as surfactant in pharmaceutical products. In particular, they are able to maintain the stability of an emulsion over time, even under different conditions such as temperature changes or exposure to air. Furthermore, they show a high compatibility with other ingredients and produce a stable and consistent emulsion.

The invention also relates to a compound of the general formula (I), (II) and (III)

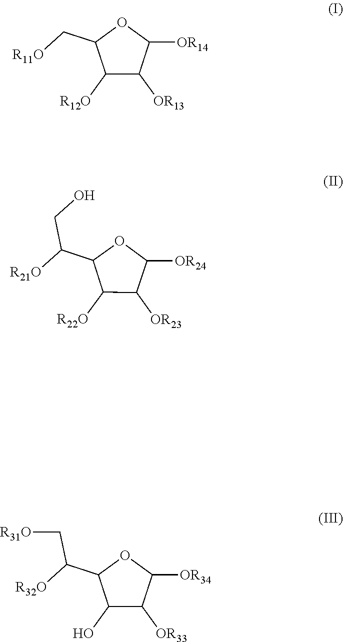

wherein R₁₁ and R₁₂ or R₂₁ and R₂₂ or R₃₁ and R₃₂ are both hydrogen or form together with CHR₅₀ a cyclic moiety or one of R₁₁ and R₁₂ or R₂₁ and R₂₂ or R₃₁ and R₃₂ is hydrogen, and the other is —CH₂R₇₀ and
wherein R₁₃ and R₁₄ or R₂₃ and R₂₄ or R₃₃ and R₃₄ are both hydrogen or form together with CHR₆₀ a cyclic moiety,
R₅₀ and R₆₀ are different from each other and are selected from the group consisting of —R₇₀, —ZR₇₀, —Z—OH, —Z—NH₂, —Z—SH, —Z—OC(O)R₇₀, —OC(O)R₇₀, —COOH, —C(O) NH₂, —C(O) NH—R₇₀, —C(O)N—(R₇₀)₂, —COOR₇₀, —Z—COOH, —Z—C(O) NH—R₇₀, —Z—C(O) NH₂, —Z—C(O)N—(R₇₀)₂, —Z—COOR₇₀, —CH(COOH)₂, —CH(COOR₇₀)₂, and —Z—SO₃—
wherein R₇₀ is selected from the group consisting of a linear or branched C₁ to C₂₀ alkyl, (C₁ to C₁₀)-alkyloxy-(C₁ to C₁₀)-alkyl, C₂ to C₁₀ alkenyl, C₆ to C₁₂ aryl, C₃ to C₁₀ cycloalkyl, cycloalkylalkyl and cycloalkylalkenyl,
wherein Z is selected from the group consisting of a linear or branched C₁ to C₁₀ alkyl, linear or branched C₃ to C₁₀ cycloalkyl, a linear or branched C₆ to C₁₀ aryl, a (C₁ to C₁₀)-alkyloxy-(C₁ to C₁₀)-alkyl, cycloalkylalkyl and cycloalkylalkenyl,
with the proviso that
not all of R₁₁, R₁₂, R₁₃ and R₁₄ or R₂₁, R₂₂, R₂₃ and R₂₄ or R₃₁, R₃₂, R₃₃ and R₃₄ are hydrogen and
if one of R₁₁, and R₁₂ or R₂₁ and R₂₂ or R₃₁ and R₃₂ is hydrogen and the other is —CH₂R₇₀, R₁₃ and R₁₄ or R₂₃ and R₂₄ or R₃₃ and R₃₄ form together with —CHR₆₀ a cyclic moiety, wherein R₆₀ is R₇₀.

One embodiment of the present invention relates to compound Ia

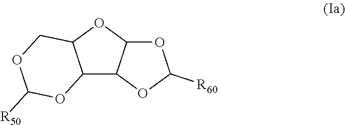

wherein R₅₀ and R₇₀ are as defined below:

| R₅₀ | R₆₀ | R₅₀ | R₆₀ |
|---|---|---|---|
| —Z—OC(O)—R₇₀ | —Z—OH | —C(O)NH—R₇₀ | —Z—OH |
| —C(O)—OR₇₀ | —Z—OH | —Z—C(O)NH—R₇₀ | —Z—OH |
| —Z—C(O)O—R₇₀ | —Z—OH | —CH(COOR₇₀)₂ | —Z—OH |
| —Z—OC(O)—R₇₀ | —COOH | —C(O)NH—R₇₀ | —COOH |
| —C(O)—OR₇₀ | —COOH | —Z—C(O)NH—R₇₀ | —COOH |
| —Z—C(O)O—R₇₀ | —COOH | —CH(COOR₇₀)₂ | —COOH |
| —Z—OC(O)—R₇₀ | —Z—COOH | —C(O)NH—R₇₀ | —Z—COOH |
| —C(O)—OR₇₀ | —Z—COOH | —Z—C(O)NH—R₇₀ | —Z—COOH |
| —Z—C(O)O—R₇₀ | —Z—COOH | —CH(COOR₇₀)₂ | —Z—COOH |
| —Z—OC(O)—R₇₀ | —CH—(COOH)₂ | —C(O)NH—R₇₀ | —CH—(COOH)₂ |
| —C(O)—OR₇₀ | —CH—(COOH)₂ | —Z—C(O)NH—R₇₀ | —CH—(COOH)₂ |
| —Z—C(O)O—R₇₀ | —CH—(COOH)₂ | —CH(COOR₇₀)₂ | —CH—(COOH)₂ |
| —Z—OH | —Z—OC(O)—R₇₀ | —Z—OH | —C(O)NH—R₇₀ |
| —Z—OH | —C(O)—OR₇₀ | —Z—OH | —Z—C(O)NH—R₇₀ |
| —Z—OH | —Z—C(O)O—R₇₀ | —Z—OH | —CH(COOR₇₀)₂ |
| —COOH | —Z—OC(O)—R₇₀ | —COOH | —C(O)NH—R₇₀ |
| —COOH | —C(O)—OR₇₀ | —COOH | —Z—C(O)NH—R₇₀ |
| —COOH | —Z—C(O)O—R₇₀ | —COOH | —CH(COOR₇₀)₂ |
| —Z—COOH | —Z—OC(O)—R₇₀ | —Z—COOH | —C(O)NH—R₇₀ |
| —Z—COOH | —C(O)—OR₇₀ | —Z—COOH | —Z—C(O)NH—R₇₀ |
| —Z—COOH | —Z—C(O)O—R₇₀ | —Z—COOH | —CH(COOR₇₀)₂ |
| —CH—(COOH)₂ | —Z—OC(O)—R₇₀ | —CH—(COOH)₂ | —C(O)NH—R₇₀ |
| —CH—(COOH)₂ | —C(O)—OR₇₀ | —CH—(COOH)₂ | —Z—C(O)NH—R₇₀ |
| —CH—(COOH)₂ | —Z—C(O)O—R₇₀ | —CH—(COOH)₂ | —CH(COOR₇₀)₂ |

One embodiment of the present invention relates to compound Ia

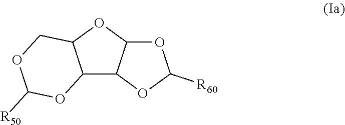

wherein R₅₀ and R₇₀ are as defined below:

| R₅₀ | R₆₀ |
|---|---|
| —(CH₂)₆CH₃ | —(CH₂)₆CH₃ |
| —(CH₂)₈CH₃ | —(CH₂)₈CH₃ |
| —(CH₂)₁₀CH₃ | —(CH₂)₁₀CH₃ |

One embodiment of the present invention relates to compound Ib

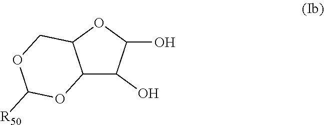

wherein R₅₀ is selected from the group consisting of —R₇₀, —Z—OC(O)—R₇₀, —C(O)—OR₇₀, —Z—C(O)O—R₇₀, —C(O)NH—R₇₀, —Z—C(O)NH—R₇₀, —CH(COOR₇₀)₂, preferably selected from the group consisting of —Z—C(O)—OR₇₀, —C(O)—OR₇₀ and —R₇₀, and most preferably selected from the group consisting of —C(O)—O(CH₂)₄CH₃, —C(O)—O(CH₂)₅CH₃, —C(O)—O(CH₂)₆CH₃, —C(O)—O(CH₂)₇CH₃, —C(O)—O(CH₂)₈CH₃, O(CH₂)₉CH₃, —C(O)—O(CH₂)₁₀CH₃, —C(O)—O(CH₂)₁₁CH₃, —C(O)—O(CH₂)₁₂CH₃, O(CH₂)₁₃CH₃, —C(O)—O(CH₂)₁₄CH₃, —CH₂—C(O)—O(CH₂)₄CH₃, —CH₂—C(O)—O(CH₂)₅CH₃, —CH₂—C(O)—O(CH₂)₆CH₃, —CH₂—C(O)—O(CH₂)₇CH₃, —CH₂—C(O)—O(CH₂)₈CH₃, —CH₂—C(O)—O(CH₂)₉CH₃, —CH₂—C(O)—O(CH₂)₁₀CH₃, —CH₂—C(O)—O(CH₂)₁₁CH₃, —CH₂—C(O)—O(CH₂)₁₂CH₃, —CH₂—C(O)—O(CH₂)₁₃CH₃, —CH₂—C(O)—O(CH₂)₁₄CH₃, —(CH₂)₂—C(O)—O(CH₂)₄CH₃, —(CH₂)₂—C(O)—O(CH₂)₅CH₃, —(CH₂)₂—C(O)—O(CH₂)₆CH₃, —(CH₂)₂—C(O)—O(CH₂)₇CH₃, —(CH₂)₂—C(O)—O(CH₂)₈CH₃, —(CH₂)₂—C(O)—O(CH₂)₉CH₃, —(CH₂)₂—C(O)—O(CH₂)₁₀CH₃, —(CH₂)₂—C(O)—O(CH₂)₁₁CH₃, —(CH₂)₂—C(O)—O(CH₂)₁₂CH₃, —(CH₂)₂—C(O)—O(CH₂)₁₅CH₃, —(CH₂)₂—C(O)—O(CH₂)₁₄CH₃, —(CH₂)₃—C(O)—O(CH₂)₄CH₃, —(CH₂)₂—C(O)—O(CH₂)₅CH₃, —(CH₂)₃—C(O)—O(CH₂)₆CH₃, —(CH₂)₂—C(O)—O(CH₂)₇CH₃, —(CH₂)₂—C(O)—O(CH₂)₈CH₃, —(CH₂)₃—C(O)—O(CH₂)₉CH₃, —(CH₂)₂—C(O)—O(CH₂)₁₀CH₃, —(CH₂)₂—C(O)—O(CH₂)₁₁CH₃, —(CH₂)₃—C(O)—O(CH₂)₁₂CH₃, —(CH₂)₃—C(O)—O(CH₂)₁₅CH₃, —(CH₂)₃—C(O)—O(CH₂)₁₄CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound Ic

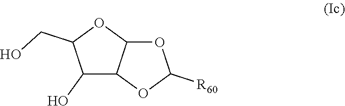

wherein R₆₀ is selected from the group consisting of —R₇₀, —Z—OC(O)—R₇₀, —C(O)—OR₇₀, —Z—C(O)O—R₇₀, —C(O)NH—R₇₀, —Z—C(O)NH—R₇₀, —CH(COOR₇₀)₂, preferably selected from the group consisting of —Z—C(O)—OR₇₀, —C(O)—OR₇₀ and —R₇₀, and most preferably selected from the group consisting of —C(O)—O(CH₂)₄CH₃, —C(O)—O(CH₂)₅CH₃, —C(O)—O(CH₂)₆CH₃, —C(O)—O(CH₂)₇CH₃, —C(O)—O(CH₂)₈CH₃, —C(O)—O(CH₂)₉CH₃, —C(O)—O(CH₂)₁₀CH₃, —C(O)—O(CH₂)₁₁CH₃, —C(O)—O(CH₂)₁₂CH₃, —C(O)—O(CH₂)₁₃CH₃, —C(O)—O(CH₂)₁₄CH₃, —CH₂—C(O)—O(CH₂)₄CH₃, —CH₂—C(O)—O(CH₂)₅CH₃, —CH₂—C(O)—O(CH₂)₆CH₃, —CH₂—C(O)—O(CH₂)₇CH₃, —CH₂—C(O)—O(CH₂)₈CH₃, —CH₂—C(O)—O(CH₂)₉CH₃, —CH₂—C(O)—O(CH₂)₁₀CH₃, —CH₂—C(O)—O(CH₂)₁₁CH₃, —CH₂—C(O)—O(CH₂)₁₂CH₃, —CH₂—C(O)—O(CH₂)₁₃CH₃, —CH₂—C(O)—O(CH₂)₁₄CH₃, —(CH₂)₂—C(O)—O(CH₂)₄CH₃, —(CH₂)₂—C(O)—O(CH₂)₅CH₃, —(CH₂)₂—C(O)—O(CH₂)₆CH₃, —(CH₂)₂—C(O)—O(CH₂)₇CH₃, —(CH₂)₂—C(O)—O(CH₂)₈CH₃, —(CH₂)₂—C(O)—O(CH₂)₉CH₃, —(CH₂)₂—C(O)—O(CH₂)₁₀CH₃, —(CH₂)₂—C(O)—O(CH₂)₁₁CH₃, —(CH₂)₂—C(O)—O(CH₂)₁₂CH₃, —(CH₂)₂—C(O)—O(CH₂)₁₃CH₃, —(CH₂)₂—C(O)—O(CH₂)₁₄CH₃, —(CH₂)₃—C(O)—O(CH₂)₄CH₃, —(CH₂)₃—C(O)—O(CH₂)₅CH₃, —(CH₂)₃—C(O)—O(CH₂)₆CH₃, —(CH₂)₃—C(O)—O(CH₂)₇CH₃, —(CH₂)₃—C(O)—O(CH₂)₈CH₃, —(CH₂)₃—C(O)—O(CH₂)₉CH₃, —(CH₂)₃—C(O)—O(CH₂)₁₀CH₃, —(CH₂)₃—C(O)—O(CH₂)₁₁CH₃, —(CH₂)₃—C(O)—O(CH₂)₁₂CH₃, —(CH₂)₃—C(O)—O(CH₂)₁₃CH₃, —(CH₂)₃—C(O)—O(CH₂)₁₄CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound Ic

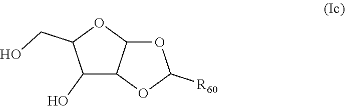

wherein R₆₀ is —R₇₀ and —R₇₀ is selected from the group consisting of —CH₃, —(CH₂) CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃ and —(CH₂)₁₉CH₃,
preferably —R₇₀ is selected from the group consisting of —(CH₂)₆CH₃, —(CH₂)₇CH₃, —(CH₂)₈CH₃, —(CH₂)₉CH₃ and —(CH₂)₁₀CH₃,
more preferably —R₇₀ is —(CH₂)₁₀CH₃.

Another aspect of the invention relates to a compound of formula (I), (II) or (III) is selected from the group consisting of

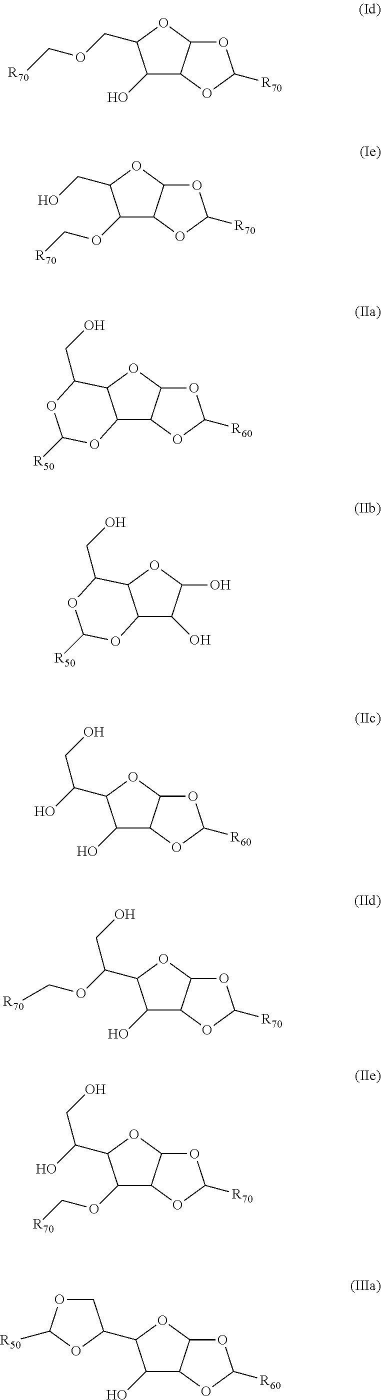

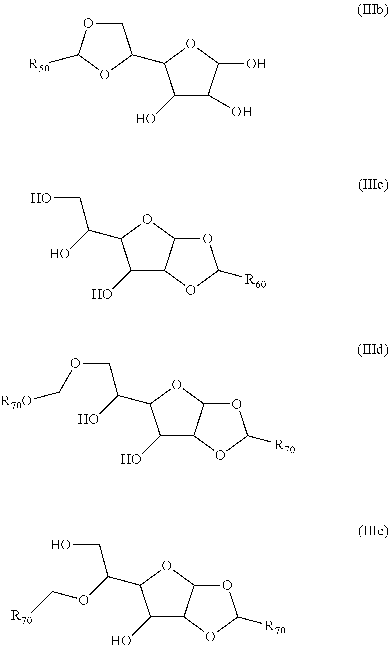

wherein R₅₀, R₆₀ and R₇₀ have the same definition as above.

In compound of formula (I), (II) or (III) preferably at least one of R₅₀ and R₆₀ is selected from the group consisting of —Z—OC(O)R₇₀, —COOR₇₀ and —Z—COOR₇₀ and the other is selected from the group consisting of —Z—OH, —COOH and —Z—COOH.

One embodiment of the present invention relates to compound Id

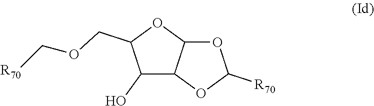

wherein R₇₀ is selected from the group consisting of —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃ and —(CH₂)₁₉CH₃,
preferably R₇₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃,
more preferably R₇₀ is selected from the group consisting of —(CH₂)₆CH₃, —(CH₂)₇CH₃, —(CH₂)₈CH₃, —(CH₂)₉CH₃ and —(CH₂)₁₀CH₃ most preferably R₇₀ is —(CH₂)₁₀CH₃.

One embodiment of the present invention relates to compound Ie

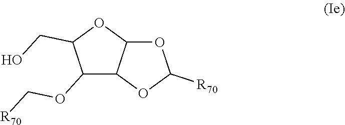

wherein R₇₀ is selected from the group consisting of —CH₃, —(CH₂)CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —(CH₂)₁₈CH₃ and —(CH₂)₁₉CH₃,
preferably R₇₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃ and —(CH₂)₁₄CH₃,
more preferably R₇₀ is selected from the group consisting of —(CH₂)₆CH₃, —(CH₂)₇CH₃, —(CH₂)₈CH₃, —(CH₂)₉CH₃ and —(CH₂)₁₀CH₃ most preferably R₇₀ is —(CH₂)₁₀CH₃.

One embodiment of the present invention relates to compound IIa

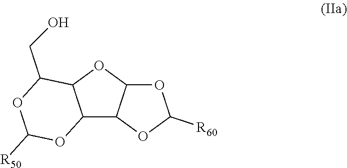

wherein R₅₀ and R₆₀ are as defined below:

| R₅₀ | R₆₀ | R₅₀ | R₆₀ |
|---|---|---|---|
| —Z—OC(O)—R₇₀ | —Z—OH | —C(O)NH—R₇₀ | —Z—OH |
| —C(O)—OR₇₀ | —Z—OH | —Z—C(O)NH—R₇₀ | —Z—OH |
| —Z—C(O)O—R₇₀ | —Z—OH | —CH(COOR₇₀)₂ | —Z—OH |
| —Z—OC(O)—R₇₀ | —COOH | —C(O)NH—R₇₀ | —COOH |
| —C(O)—OR₇₀ | —COOH | —Z—C(O)NH—R₇₀ | —COOH |
| —Z—C(O)O—R₇₀ | —COOH | —CH(COOR₇₀)₂ | —COOH |
| —Z—OC(O)—R₇₀ | —Z—COOH | —C(O)NH—R₇₀ | —Z—COOH |
| —C(O)—OR₇₀ | —Z—COOH | —Z—C(O)NH—R₇₀ | —Z—COOH |
| —Z—C(O)O—R₇₀ | —Z—COOH | —CH(COOR₇₀)₂ | —Z—COOH |
| —Z—OC(O)—R₇₀ | —CH—(COOH)₂ | —C(O)NH—R₇₀ | —CH—(COOH)₂ |
| —C(O)—OR₇₀ | —CH—(COOH)₂ | —Z—C(O)NH—R₇₀ | —CH—(COOH)₂ |
| —Z—C(O)O—R₇₀ | —CH—(COOH)₂ | —CH(COOR₇₀)₂ | —CH—(COOH)₂ |
| —Z—OH | —Z—OC(O)—R₇₀ | —Z—OH | —C(O)NH—R₇₀ |
| —Z—OH | —C(O)—OR₇₀ | —Z—OH | —Z—C(O)NH—R₇₀ |
| —Z—OH | —Z—C(O)O—R₇₀ | —Z—OH | —CH(COOR₇₀)₂ |
| —COOH | —Z—OC(O)—R₇₀ | —COOH | —C(O)NH—R₇₀ |
| —COOH | —C(O)—OR₇₀ | —COOH | —Z—C(O)NH—R₇₀ |
| —COOH | —Z—C(O)O—R₇₀ | —COOH | —CH(COOR₇₀)₂ |
| —Z—COOH | —Z—OC(O)—R₇₀ | —Z—COOH | —C(O)NH—R₇₀ |
| —Z—COOH | —C(O)—OR₇₀ | —Z—COOH | —Z—C(O)NH—R₇₀ |
| —Z—COOH | —Z—C(O)O—R₇₀ | —Z—COOH | —CH(COOR₇₀)₂ |
| —CH—(COOH)₂ | —Z—OC(O)—R₇₀ | —CH—(COOH)₂ | —C(O)NH—R₇₀ |
| —CH—(COOH)₂ | —C(O)—OR₇₀ | —CH—(COOH)₂ | —Z—C(O)NH—R₇₀ |
| —CH—(COOH)₂ | —Z—C(O)O—R₇₀ | —CH—(COOH)₂ | —CH(COOR₇₀)₂ |

One embodiment of the present invention relates to compound IIb

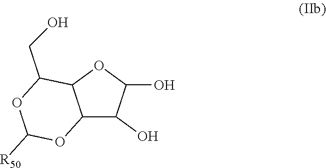

wherein R₅₀ is selected from the group consisting of —Z—OC(O)R₇₀, —COOR₇₀ and —Z—COOR₇₀, most preferably selected from the group consisting of —CH₂—OC(O)(CH₂)₄CH₃, —CH₂—OC(O)(CH₂)₅CH₃, —CH₂—OC(O)(CH₂)₆CH₃, —CH₂—OC(O)(CH₂)₇CH₃, —CH₂—OC(O)(CH₂)₈CH₃, —CH₂—OC(O)(CH₂)₉CH₃, —CH₂—OC—CH₂—(O)(CH₂)₁₀CH₃, OC(O)(CH₂)₁₁CH₃, —CH₂—OC(O)(CH₂)₁₂CH₃, —CH₂—OC(O)(CH₂)₁₃CH₃, —CH₂—OC(O)(CH₂)₁₄CH₃, —(CH₂)₂—OC(O)(CH₂)₄CH₃, —(CH₂)₂—OC(O)(CH₂)₅CH₃, —(CH₂)₂—OC(O)(CH₂)₆CH₃, —(CH₂)₂—OC(O)(CH₂)₇CH₃, —(CH₂)₂—OC(O)(CH₂)₈CH₃, —(CH₂)₂—OC(O)(CH₂)₈CH₃, —(CH₂)₂—OC(O)(CH₂)₉CH₃, —(CH₂)₂—OC(O)(CH₂)₁₀CH₃, —(CH₂)₂—OC(O)(CH₂)₁₁CH₃, —(CH₂)₂—OC(O)(CH₂)₁₂CH₃, —(CH₂)₂—OC(O)(CH₂)₁₃CH₃, —(CH₂)₂—OC(O)(CH₂)₁₂CH₃, —(CH₂)₃—OC(O)(CH₂)₄CH₃, —(CH₂)₃—OC(O)(CH₂)₅CH₃, —(CH₂)₃—OC(O)(CH₂)₆CH₃, —(CH₂) OC(O)(CH₂)₇CH₃, —(CH₂)₃—OC(O)(CH₂)₈CH₃, —(CH₂)₃—OC(O)(CH₂)₈CH₃, —(CH₂)₃—OC(O)(CH₂)₉CH₃, —(CH₂)₃—OC(O)(CH₂)₁₀CH₃, —(CH₂)₃—OC(O)(CH₂)₁₁CH₃, —(CH₂)₃—OC(O)(CH₂)₁₂CH₃, —(CH₂)₃—OC(O)(CH₂)₁₃CH₃, —(CH₂)₃—OC(O)(CH₂)₁₂CH₃, —(CH₂) OC(O)(CH₂)₄CH₃, —(CH₂)₄—OC(O)(CH₂)₅CH₃, —(CH₂)₄—OC(O)(CH₂)₆CH₃, —(CH₂)₄—OC(O)(CH₂)₇CH₃, —(CH₂)₄—OC(O)(CH₂)₈CH₃, —(CH₂)₄—OC(O)(CH₂)₈CH₃, —(CH₂)₄—OC(O)(CH₂)₉CH₃, —(CH₂)₄—OC(O)(CH₂)₁₀CH₃, —(CH₂)₄—OC(O)(CH₂)₁₁CH₃, —(CH₂)₄—OC(O)(CH₂)₁₂CH₃, —(CH₂)₄—OC(O)(CH₂)₁₃CH₃, —(CH₂)₄—OC(O)(CH₂)₁₂CH₃, —(CH₂)₅—OC(O)(CH₂)₄CH₃, —(CH₂)₅—OC(O)(CH₂)₅CH₃, —(CH₂)₅—OC(O)(CH₂)₆CH₃, —(CH₂)₅—OC(O)(CH₂)CH₇, —(CH₂)₅—OC(O)(CH₂)₈CH₃, —(CH₂)₅—OC(O)(CH₂)₈CH₃, —(CH₂)₅—OC(O)(CH₂)₉CH₃, —(CH₂)₅—OC(O)(CH₂)₁₀CH₃, —(CH₂)₅—OC(O)(CH₂)₁₁CH₃, —(CH₂)₂—OC(O)(CH₂)₁₂CH₃, —(CH₂)₅—OC(O)(CH₂)₁₃CH₃, —(CH₂)₅—OC(O)(CH₂)₁₂CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₀CH₃, —COO(CH₂)₁₁CH₃, —COO(CH₂)₁₂CH₃, —COO(CH₂)₁₃CH₃, —COO(CH₂)₁₄CH₃, —CH₂—C(O)O(CH₂)₄CH₃, —CH₂—C(O)O(CH₂)₅CH₃, —CH₂—C(O)O(CH₂)₆CH₃, —CH₂—C(O)O(CH₂)₇CH₃, —CH₂—C(O)O(CH₂)₈CH₃, —CH₂—C(O)O(CH₂)₉CH₃, —CH₂—C(O)O(CH₂)₁₀CH₃, —CH₂—C(O)O(CH₂)₁₁CH₃, —CH₂—C(O)O(CH₂)₁₂CH₃, —CH₂—C(O)O(CH₂)₁₃CH₃, —CH₂—C(O)O(CH₂)₁₄CH₃, —(CH₂)₂—C(O)O(CH₂)₄CH₃, —(CH₂)₂—C(O)OCH₂)₅CH₃, —(CH₂)₂—C(O)OCH₂)₆CH₃, —(CH₂)₂—C(O)OCH₂)₇CH₃, —(CH₂)₂—C(O)O(CH₂)₈CH₃, —(CH₂)₂—C(O)O(CH₂)₈CH₃, —(CH₂)₂—C(O)O(CH₂)₉CH₃, —(CH₂)₂—C(O)OCH₂)₁₀CH₃, —(CH₂)₂—C(O)O (CH₂)₁₁CH₃, —(CH₂)₂—C(O)OCH₂)₁₂CH₃, —(CH₂)₂—C(O)O(CH₂)₁₃CH₃, —(CH₂)₂—C(O)O(CH₂)₁₂CH₃, —(CH₂)₃—C(O)O(CH₂)₄CH₃, —(CH₂)₃—C(O)O(CH₂)₅CH₃, —(CH₂)₃—C(O)O(CH₂)₆CH₃, —(CH₂)₃—C(O)O(CH₂)₇CH₃, —(CH₂)₃—C(O)O(CH₂)₈CH₃, —(CH₂)₃—C(O)O(CH₂)₈CH₃, —(CH₂)₃—C(O)O(CH₂)₉CH₃, —(CH₂)₃—C(O)O(CH₂)₁₀CH₃, —(CH₂)₃—C(O)O(CH₂)₁₁CH₃, —(CH₂)₃—C(O)O(CH₂)₁₂CH₃, —(CH₂)₃—C(O)OCH₂)₁₃CH₃, —(CH₂)₂—C(O)O(CH₂)₁₂CH₃, —(CH₂)₄—C(O)O(CH₂)₄CH₃, —(CH₂)₄—C(O)O(CH₂)₅CH₃, —(CH₂)₄—C(O)O(CH₂)₆CH₃, —(CH₂)₄—C(O)O(CH₂)₇CH₃, —(CH₂)₄—C(O)O(CH₂)₈CH₃, —(CH₂)₄—C(O)O(CH₂)₈CH₃, —(CH₂)₄—C(O)O(CH₂)₉CH₃, —(CH₂)₄—C(O)O(CH₂)₁₀CH₃, —(CH₂)₄—C(O)O(CH₂)₁₁CH₃, —(CH₂)₄—C(O)O(CH₂)₁₂CH₃, —(CH₂)₄—C(O)O(CH₂)₁₃CH₃, —(CH₂)₄—C(O)O(CH₂)₁₂CH₃, —(CH₂)₅—C(O)O(CH₂)₄CH₃, —(CH₂)₅—C(O)O(CH₂)₅CH₃, —(CH₂)₅—C(O)O(CH₂)₆CH₃, —(CH₂)₅—C(O)O(CH₂)₇CH₃, —(CH₂)₅—C(O)O(CH₂)₈CH₃, —(CH₂)₅—C(O)OCH₂)₈CH₃, —(CH₂)₅—C(O)O(CH₂)₉CH₃, —(CH₂)₅—C(O)O(CH₂)₁₀CH₃, —(CH₂)₅—C(O)O(CH₂)₁₁CH₃, —(CH₂)₂—C(O)O(CH₂)₁₂CH₃, —(CH₂)₅—C(O)O(CH₂)₁₃CH₃, and —(CH₂)₅—C(O)O(CH₂)₁₂CH₃.

One embodiment of the present invention relates to compound IIc,

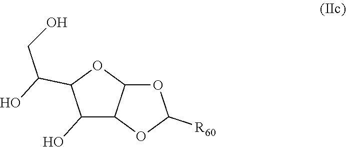

wherein R₆₀ is selected from the group consisting of —R₇₀, —Z—OC(O)—R₇₀, —C(O)—OR₇₀, —Z—C(O)O—R₇₀, —C(O)NH—R₇₀, —Z—C(O)NH—R₇₀ and —CH(COOR₇₀)₂, preferably selected from the group consisting of —Z—C(O)—OR₇₀, —C(O)—OR₇₀ and —R₇₀, and most preferably selected from the group consisting of —C(O)—O(CH₂)₄CH₃, —C(O)—O(CH₂)₅CH₃, —C(O)—O(CH₂)₆CH₃, —C(O)—O(CH₂)₇CH₃, —C(O)—O(CH₂)₈CH₃, —C(O)—O(CH₂)₉CH₃, —C(O)—O(CH₂)₁₀CH₃, —C(O)—O(CH₂)₁₁CH₃, —C(O)—O(CH₂)₁₂CH₃, —C(O)—O(CH₂)₁₃CH₃, —C(O)—O(CH₂)₁₄CH₃, —CH₂—C(O)—O(CH₂)₄CH₃, —CH₂—C(O)—O(CH₂)₅CH₃, —CH₂—C(O)—O(CH₂)₆CH₃, —CH₂—C(O)—O(CH₂)₇CH₃, —CH₂—C(O)—O(CH₂)₈CH₃, —CH₂—C(O)—O(CH₂)₉CH₃, —CH₂—C(O)—O(CH₂)₁₀CH₃, —CH₂—C(O)—O(CH₂)₁₁CH₃, —CH₂—C(O)—O(CH₂)₁₂CH₃, —CH₂—C(O)—O(CH₂)₁₃CH₃, —CH₂—C(O)—O(CH₂)₁₄CH₃, —(CH₂)₂—C(O)—O(CH₂)₄CH₃, —(CH₂)₂—C(O)—O(CH₂)₅CH₃, —(CH₂)₂—C(O)—O(CH₂)₆CH₃, —(CH₂)₂—C(O)—O(CH₂)₇CH₃, —(CH₂)₂—C(O)—O(CH₂)₈CH₃, —(CH₂)₂—C(O)—O(CH₂)₉CH₃, —(CH₂)₂—C(O)—O(CH₂)₁₀CH₃, —(CH₂)₂—C(O)—O(CH₂)₁₁CH₃, —(CH₂)₂—C(O)—O(CH₂)₁₂CH₃, —(CH₂)₂—C(O)—O(CH₂)₁₃CH₃, —(CH₂)₂—C(O)—O(CH₂)₁₄CH₃, —(CH₂)₃—C(O)—O(CH₂)₄CH₃, —(CH₂)₂—C(O)—O(CH₂)₅CH₃, —(CH₂)₃—C(O)—O(CH₂)₆CH₃, —(CH₂)₃—C(O)—O(CH₂)₇CH₃, —(CH₂)₃—C(O)—O(CH₂)₈CH₃, —(CH₂)₃—C(O)—O(CH₂)₉CH₃, —(CH₂)₃—C(O)—O(CH₂)₁₀CH₃, —(CH₂)₃—C(O)—O(CH₂)₁₁CH₃, —(CH₂)₃—C(O)—O(CH₂)₁₂CH₃, —(CH₂)₃—C(O)—O(CH₂)₁₃CH₃, —(CH₂)₃—C(O)—O(CH₂)₁₄CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound IId,

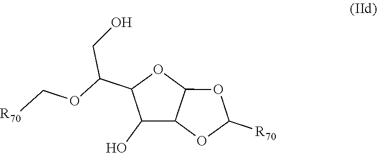

wherein R₇₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound IIe,

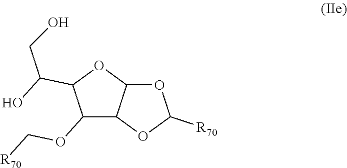

wherein R₁₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₅CH₃, and —(CH₂)₁₄CH₃.

One embodiment of the present invention relates to compound IIIa,

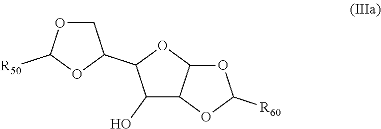

wherein R₅₀ and R₆₀ are as defined below:

| R₅₀ | R₆₀ | R₅₀ | R₆₀ |
|---|---|---|---|
| —Z—OC(O)—R₇₀ | —Z—OH | —C(O)NH—R₇₀ | —Z—OH |
| —C(O)—OR₇₀ | —Z—OH | —Z—C(O)NH—R₇₀ | —Z—OH |
| —Z—C(O)O—R₇₀ | —Z—OH | —CH(COOR₇₀)₂ | —Z—OH |
| —Z—OC(O)—R₇₀ | —COOH | —C(O)NH—R₇₀ | —COOH |
| —C(O)—OR₇₀ | —COOH | —Z—C(O)NH—R₇₀ | —COOH |
| —Z—C(O)O—R₇₀ | —COOH | —CH(COOR₇₀)₂ | —COOH |
| —Z—OC(O)—R₇₀ | —Z—COOH | —C(O)NH—R₇₀ | —Z—COOH |
| —C(O)—OR₇₀ | —Z—COOH | —Z—C(O)NH—R₇₀ | —Z—COOH |
| —Z—C(O)O—R₇₀ | —Z—COOH | —CH(COOR₇₀)₂ | —Z—COOH |
| —Z—OC(O)—R₇₀ | —CH—(COOH)₂ | —C(O)NH—R₇₀ | —CH—(COOH)₂ |
| —C(O)—OR₇₀ | —CH—(COOH)₂ | —Z—C(O)NH—R₇₀ | —CH—(COOH)₂ |
| —Z—C(O)O—R₇₀ | —CH—(COOH)₂ | —CH(COOR₇₀)₂ | —CH—(COOH)₂ |
| —Z—OH | —Z—OC(O)—R₇₀ | —Z—OH | —C(O)NH—R₇₀ |
| —Z—OH | —C(O)—OR₇₀ | —Z—OH | —Z—C(O)NH—R₇₀ |
| —Z—OH | —Z—C(O)O—R₇₀ | —Z—OH | —CH(COOR₇₀)₂ |
| —COOH | —Z—OC(O)—R₇₀ | —COOH | —C(O)NH—R₇₀ |
| —COOH | —C(O)—OR₇₀ | —COOH | —Z—C(O)NH—R₇₀ |
| —COOH | —Z—C(O)O—R₇₀ | —COOH | —CH(COOR₇₀)₂ |
| —Z—COOH | —Z—OC(O)—R₇₀ | —Z—COOH | —C(O)NH—R₇₀ |
| —Z—COOH | —C(O)—OR₇₀ | —Z—COOH | —Z—C(O)NH—R₇₀ |
| —Z—COOH | —Z—C(O)O—R₇₀ | —Z—COOH | —CH(COOR₇₀)₂ |
| —CH—(COOH)₂ | —Z—OC(O)—R₇₀ | —CH—(COOH)₂ | —C(O)NH—R₇₀ |
| —CH—(COOH)₂ | —C(O)—OR₇₀ | —CH—(COOH)₂ | —Z—C(O)NH—R₇₀ |
| —CH—(COOH)₂ | —Z—C(O)O—R₇₀ | —CH—(COOH)₂ | —CH(COOR₇₀)₂ |

One embodiment of the present invention relates to compound IIIb,

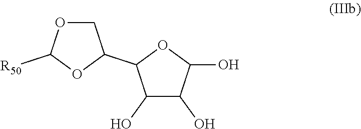

wherein R₅₀ is selected from the group consisting of —R₇₀, —Z—OC(O)R₁₀, —COOR₇₀ and —Z—COOR₇₀, most preferably selected from the group consisting of —CH₂—OC(O)(CH₂)₄CH₃, —CH₂—OC(O)(CH₂)₅CH₃, —CH₂—OC(O)(CH₂)₆CH₃, —CH₂—OC(O)(CH₂)₇CH₃, —CH₂—OC(O)(CH₂)₈CH₃, —CH₂—OC(O)(CH₂)₉CH₃, —CH₂—OC(O)(CH₂)₁₀CH₃, —CH₂—OC(O)(CH₂)₁₁CH₃, —CH₂—OC(O)(CH₂)₁₂CH₃, —CH₂—OC(O)(CH₂)₁₃CH₃, —CH₂—OC(O)(CH₂)₁₄CH₃, —(CH₂)₂—OC(O)(CH₂)₄CH₃, —(CH₂)₂—OC(O)(CH₂)₅CH₃, —(CH₂)₂—OC(O)(CH₂)₆CH₃, —(CH₂)₂—OC(O)(CH₂)₇CH₃, —(CH₂)₂—OC(O)(CH₂)₈CH₃, —(CH₂)₂—OC(O)(CH₂)₈CH₃, —(CH₂)₂—OC(O)(CH₂)₉CH₃, —(CH₂)₂—OC(O)(CH₂)₁₀CH₃, —(CH₂)₂—OC(O)(CH₂)₁₁CH₃, —(CH₂)₂—OC(O)(CH₂)₁₂CH₃, —(CH₂)₂—OC(O)(CH₂)₁₃CH₃, —(CH₂)₂—OC(O)(CH₂)₁₂CH₃, —(CH₂)₃—OC(O)(CH₂)₄CH₃, —(CH₂)₃—OC(O)(CH₂)₅CH₃, —(CH₂)₃—OC(O)(CH₂)₆CH₃, —(CH₂)₃—OC(O)(CH₂)₇CH₃, —(CH₂)₃—OC(O)(CH₂)₈CH₃, —(CH₂)₃—OC(O)(CH₂)₈CH₃, —(CH₂)₃—OC(O)(CH₂)₉CH₃, —(CH₂)₃—OC(O)(CH₂)₁₀CH₃, —(CH₂)₃—OC(O)(CH₂)₁₁CH₃, —(CH₂)₃—OC(O)(CH₂)₁₂CH₃, —(CH₂)₃—OC(O)(CH₂)₁₃CH₃, —(CH₂)₃—OC(O)(CH₂)₁₂CH₃, —(CH₂)₄—OC(O)(CH₂)₄CH₃, —(CH₂)₄—OC(O)(CH₂)₅CH₃, —(CH₂)₄—OC(O)(CH₂)₆CH₃, —(CH₂)₄—OC(O)(CH₂)₇CH₃, —(CH₂)₄—OC(O)(CH₂)₈CH₃, —(CH₂)₄—OC(O)(CH₂)₈CH₃, —(CH₂)₄—OC(O)(CH₂)₉CH₃, —(CH₂)₄—OC(O)(CH₂)₁₀CH₃, —(CH₂)₄—OC(O)(CH₂)₁₁CH₃, —(CH₂)₄—OC(O)(CH₂)₁₂CH₃, —(CH₂)₄—OC(O)(CH₂)₁₃CH₃, —(CH₂)₄—OC(O)(CH₂)₁₂CH₃, —(CH₂)₅—OC(O)(CH₂)₄CH₃, —(CH₂)₅—OC(O)(CH₂)₅CH₃, —(CH₂)₅—OC(O)(CH₂)₆CH₃, —(CH₂)₅—OC(O)(CH₂)₇CH₃, —(CH₂)₅—OC(O)(CH₂)₈CH₃, —(CH₂)₅—OC(O)(CH₂)₈CH₃, —(CH₂)₅—OC(O)(CH₂)₉CH₃, —(CH₂)₅—OC(O)(CH₂)₁₀CH₃, —(CH₂)₅—OC(O)(CH₂)₁₁CH₃, —(CH₂)₂—OC(O)(CH₂)₁₂CH₃, —(CH₂)₅—OC(O)(CH₂)₁₃CH₃, —(CH₂)₅—OC(O)(CH₂)₁₂CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₀CH₃, —COO(CH₂)₁₁CH₃, —COO(CH₂)₁₂CH₃, —COO(CH₂)₁₃CH₃, —COO(CH₂)₁₄CH₃, —CH₂—C(O)O(CH₂)₄CH₃, —CH₂—C(O)O(CH₂)₅CH₃, —CH₂—C(O)O(CH₂)₆CH₃, —CH₂—C(O)O(CH₂)₇CH₃, —CH₂—C(O)O(CH₂)₈CH₃, —CH₂—C(O)O(CH₂)₉CH₃, —CH₂—C(O)O(CH₂)₁₀CH₃, —CH₂—C(O)O(CH₂)₁₁CH₃, —CH₂—C(O)O(CH₂)₁₂CH₃, —CH₂—C(O)O(CH₂)₁₃CH₃, —CH₂—C(O)O(CH₂)₁₄CH₃, —(CH₂)₂—C(O)O(CH₂)₄CH₃, —(CH₂)₂—C(O)OCH₂)₅CH₃, —(CH₂)₂—C(O)OCH₂)₆CH₃, —(CH₂)₂—C(O)OCH₂)₇CH₃, —(CH₂)₂—C(O)O(CH₂)₈CH₃, —(CH₂)₂—C(O)O(CH₂)₈CH₃, —(CH₂)₂—C(O)O(CH₂)₉CH₃, —(CH₂)₂—C(O)OCH₂)₁₀CH₃, —(CH₂)₂—C(O)O(CH₂)₁₁CH₃, —(CH₂)₂—C(O)OCH₂)₁₂CH₃, —(CH₂)₂—C(O)O(CH₂)₁₃CH₃, —(CH₂)₂—C(O)O(CH₂)₁₂CH₃, —(CH₂)₃—C(O)O(CH₂)₄CH₃, —(CH₂)₃—C(O)O(CH₂)₅CH₃, —(CH₂)₃—C(O)O(CH₂)₆CH₃, —(CH₂)₃—C(O)O(CH₂)₇CH₃, —(CH₂)₃—C(O)O(CH₂)₈CH₃, —(CH₂)₃—C(O)O(CH₂)₈CH₃, —(CH₂)₃—C(O)O(CH₂)₉CH₃, —(CH₂)₃—C(O)O(CH₂)₁₀CH₃, —(CH₂)₃—C(O)O(CH₂)₁₁CH₃, —(CH₂)₃—C(O)O(CH₂)₁₂CH₃, —(CH₂)₃—C(O)OCH₂)₁₃CH₃, —(CH₂)₃—C(O)O(CH₂)₁₂CH₃, —(CH₂)₄—C(O)O(CH₂)₄CH₃, —(CH₂)₄—C(O)O(CH₂)₅CH₃, —(CH₂)₄—C(O)O(CH₂)₆CH₃, —(CH₂)₄—C(O)O(CH₂)₇CH₃, —(CH₂)₄—C(O)O(CH₂)₈CH₃, —(CH₂)₄—C(O)O(CH₂)₈CH₃, —(CH₂)₄—C(O)O(CH₂)₉CH₃, (CH₂)₄—C(O)O(CH₂)₁₀CH₃, —(CH₂)₄—C(O)O(CH₂)₁₁CH₃, —(CH₂)₄—C(O)O(CH₂)₁₂CH₃, —(CH₂)₄—C(O)O (CH₂)₁₃CH₃, —(CH₂)₄—C(O)O(CH₂)₁₂CH₃, —(CH₂)₅—C(O)O(CH₂)₄CH₃, —(CH₂) C(O)O(CH₂)₅CH₃, —(CH₂)₅—C(O)O(CH₂)₆CH₃, —(CH₂)₅—C(O)O(CH₂)₇CH₃, —(CH₂)₅—C(O)O(CH₂)₈CH₃, —(CH₂)₅—C(O)OCH₂)₈CH₃, —(CH₂)₅—C(O)O(CH₂)₉CH₃, —(CH₂)₅—C(O)O(CH₂)₁₀CH₃, —(CH₂)₅—C(O)O(CH₂)₁₁CH₃, —(CH₂)₂—C(O)O(CH₂)₁₂CH₃, —(CH₂)₅—C(O)O(CH₂)₁₃CH₃ and —(CH₂)₅—C(O)O(CH₂)₁₂CH₃.

One embodiment of the present invention relates to compound IIIc,

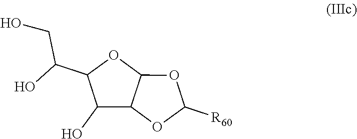

wherein R₆₀ is selected from the group consisting of —R₇₀, —Z—OC(O)—R₇₀, —C(O)—OR₇₀, —Z—C(O)O—R₇₀, —C(O)NH—R₇₀, —Z—C(O)NH—R₇₀, —CH(COOR₇₀)₂, preferably selected from the group consisting of —Z—C(O)—OR₇₀, —C(O)—OR₇₀ and —R₇₀, and most preferably selected from the group consisting of —C(O)—O(CH₂)₄CH₃, —C(O)—O(CH₂)₅CH₃, —C(O)—O(CH₂)₆CH₃, —C(O)—O(CH₂)₇CH₃, —C(O)—O(CH₂)₈CH₃, —C(O)—O(CH₂)₉CH₃, —C(O)—O(CH₂)₁₀CH₃, —C(O)—O(CH₂)₁₁CH₃, —C(O)—O(CH₂)₁₂CH₃, —C(O)—O(CH₂)₁₃CH₃, —C(O)—O(CH₂)₁₄CH₃, —CH₂—C(O)—O(CH₂)₄CH₃, —CH₂—C(O)—O(CH₂)₅CH₃, —CH₂—C(O)—O(CH₂)₆CH₃, —CH₂—C(O)—O(CH₂)₇CH₃, —CH₂—C(O)—O(CH₂)₈CH₃, —CH₂—C(O)—O(CH₂)₉CH₃, —CH₂—C(O)—O(CH₂)₁₀CH₃, —CH₂—C(O)—O(CH₂)₁₁CH₃, —CH₂—C(O)—O(CH₂)₁₂CH₃, —CH₂—C(O)—O(CH₂)₁₃CH₃, —CH₂—C(O)—O(CH₂)₁₄CH₃, —(CH₂)₂—C(O)—O(CH₂)₄CH₃, —(CH₂)₂—C(O)—O(CH₂)₅CH₃, —(CH₂)₂—C(O)—O(CH₂)₆CH₃, —(CH₂)₂—C(O)—O(CH₂)₇CH₃, —(CH₂)₂—C(O)—O(CH₂)₈CH₃, —(CH₂)₂—C(O)—O(CH₂)₉CH₃, —(CH₂)₂—C(O)—O(CH₂)₁₀CH₃, —(CH₂)₂—C(O)—O(CH₂)₁₁CH₃, —(CH₂)₂—C(O)—O(CH₂)₁₂CH₃, —(CH₂)₂—C(O)—O(CH₂)₁₃CH₃, —(CH₂)₂—C(O)—O(CH₂)₁₄CH₃, —(CH₂)₃—C(O)—O(CH₂)₄CH₃, —(CH₂)₃—C(O)—O(CH₂)₅CH₃, —(CH₂)₃—C(O)—O(CH₂)₆CH₃, —(CH₂)₃—C(O)—O(CH₂)₇CH₃, —(CH₂)₃—C(O)—O(CH₂)₈CH₃, —(CH₂)₃—C(O)—O(CH₂)₉CH₃, —(CH₂)₃—C(O)—O(CH₂)₁₀CH₃, —(CH₂)₃—C(O)—O(CH₂)₁₁CH₃, —(CH₂)₃—C(O)—O(CH₂)₁₂CH₃, —(CH₂)₃—C(O)—O(CH₂)₁₃CH₃, —(CH₂)₃—C(O)—O(CH₂)₁₄CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound IIId,

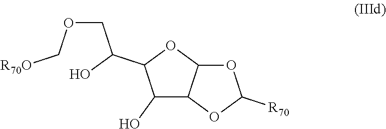

wherein R₇₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to compound IIIe,

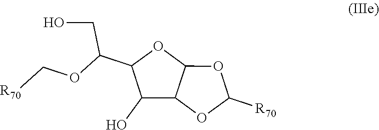

wherein R₇₀ is selected from the group consisting of —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, and —(CH₂)₁₄CH₃.

One embodiment of the present invention relates to a compound of the general formula (I), (II) and (III) wherein R₁₁ and R₁₂ or R₂₁ and R₂₂ or R₃₁ and R₃₂ are both hydrogen or form together with CHR₅₀ a cyclic moiety, or wherein R₁₃ and R₁₄ or R₂₃ and R₂₄ or R₃₃ and R₃₄ are both hydrogen or form together with CHR₆₀ a cyclic moiety, with the proviso that not all of R₁₁, R₁₂, R₁₃ and R₁₄ or R₂₁, R₂₂, R₂₃ and R₂₄ or R₃₁, R₃₂, R₃₃ and R₃₄ are hydrogen, wherein R₅₀ or R₆₀ are a linear C₁ to C₁₀ alkyl. Preferably, said compound is selected from the group consisting of compound Ia, Ib, Ic, IIa, IIb, IIc, IIIa, IIIb and IIIc, wherein R₅₀ or R₆₀ are a linear C₁ to C₁₀ alkyl, most preferably C₅ to C₁₀ alkyl.

One embodiment of the present invention relates to a compound of the general formula (I), (II) and (III) wherein R₁₁ and R₁₂ or R₂₁ and R₂₂ or R₃₁ and R₃₂ form together with CHR₅₀ a cyclic moiety and wherein R₁₃ and R₁₄ or R₂₃ and R₂₄ or R₃₃ and R₃₄ form together with CHR₆₀ a cyclic moiety, and R₅₀ and R₆₀ are different and are selected from the group consisting of —COOH and —C(O) NH—R₇₀, and wherein —R₇₀ is preferably a linear or branched C₁ to C₂₀ alkyl, more preferably a linear C₅ to C₁₅ alkyl and most preferably a linear C₁₀ to C₁₅ alkyl. Preferably, said compound is selected from the group consisting of compound Ia, IIa and IIIa, wherein R₅₀ and R₆₀ are defined as follows:

| R₅₀ | R₆₀ | R₅₀ | R₆₀ |
|---|---|---|---|
| —COOH | —C(O)NH—CH₃ | —C(O)NH—CH₃ | —COOH |
| —COOH | —C(O)NH—(CH₂)—CH₃ | —C(O)NH—(CH₂)—CH₃ | —COOH |
| —COOH | —C(O)NH—(CH₂)₂—CH₃ | —C(O)NH—(CH₂)₂—CH₃ | —COOH |
| —COOH | —C(O)NH—(CH₂)₃—CH₃ | —C(O)NH—(CH₂)₃—CH₃ | —COOH |
| —COOH | —C(O)NH—(CH₂)₄—CH₃ | —C(O)NH—(CH₂)₄—CH₃ | —COOH |
| —COOH | —C(O)NH—(CH₂)₅—CH₃ | —C(O)NH—(CH₂)₅—CH₃ | —COOH |
| —COOH | —C(O)NH—(CH₂)₆—CH₃ | —C(O)NH—(CH₂)₆—CH₃ | —COOH |
| —COOH | —C(O)NH—(CH₂)₇—CH₃ | —C(O)NH—(CH₂)₇—CH₃ | —COOH |
| —COOH | —C(O)NH—(CH₂)₈—CH₃ | —C(O)NH—(CH₂)₈—CH₃ | —COOH |
| —COOH | —C(O)NH—(CH₂)₉—CH₃ | —C(O)NH—(CH₂)₉—CH₃ | —COOH |
| —COOH | —C(O)NH—(CH₂)₁₀—CH₃ | —C(O)NH—(CH₂)₁₀—CH₃ | —COOH |
| —COOH | —C(O)NH—(CH₂)₁₁—CH₃ | —C(O)NH—(CH₂)₁₁—CH₃ | —COOH |
| —COOH | —C(O)NH—(CH₂)₁₂—CH₃ | —C(O)NH—(CH₂)₁₂—CH₃ | —COOH |
| —COOH | —C(O)NH—(CH₂)₁₃—CH₃ | —C(O)NH—(CH₂)₁₃—CH₃ | —COOH |
| —COOH | —C(O)NH—(CH₂)₁₄—CH₃ | —C(O)NH—(CH₂)₁₄—CH₃ | —COOH |
| —COOH | —C(O)NH—(CH₂)₁₅—CH₃ | —C(O)NH—(CH₂)₁₅—CH₃ | —COOH |
| —COOH | —C(O)NH—(CH₂)₁₆—CH₃ | —C(O)NH—(CH₂)₁₆—CH₃ | —COOH |
| —COOH | —C(O)NH—(CH₂)₁₇—CH₃ | —C(O)NH—(CH₂)₁₇—CH₃ | —COOH |
| —COOH | —C(O)NH—(CH₂)₁₈—CH₃ | —C(O)NH—(CH₂)₁₈—CH₃ | —COOH |
| —COOH | —C(O)NH—(CH₂)₁₉—CH₃ | —C(O)NH—(CH₂)₁₉—CH₃ | —COOH |

One embodiment of the present invention relates to a compound of the general formula (I), (II) and (III), preferably a compound of formula Ia, Ib, Ic, IIa, IIb, IIc, IIIa, IIIb, and IIIc wherein one of R₅₀ and R₆₀ is selected from the group consisting of —Z—OC(O)R₁₀, —COOR₇₀ and —Z—COOR₇₀ and the other, if present, is selected from the group consisting of —Z—OH, —COOH and —Z—COOH, and Z is a linear or branched C₁ to C₁₀ alkyl, preferably a linear C₁ to C₅ alkyl, most preferably a linear C₁ to C₃ alkyl, and wherein —R₇₀ is a linear or branched C₁ to C₂₀ alkyl, preferably a linear C₅ to Cis alkyl, most preferably a linear C₁₀ to C₁₅ alkyl.

One embodiment of the present invention relates to a compound of the general formula (I), (II) and (III), preferably a compound of formula Ia, Ib, Ic, IIa, IIb, IIc, IIIa, IIIb, and IIIc, wherein R₅₀ or R₆₀ is selected from the group consisting of —C(O)NH—R₇₀ and Z—C(O) NH—R₇₀ and R₇₀ is a linear or branched C₁ to C₂₀ alkyl, preferably a linear C₅ to C₁₅ alkyl, most preferably a linear C₁₀ to C₁₅ alkyl. Said compounds are resistant towards the hydrolysis in alkaline media and provide antistatic properties. In addition, they are usually less aggressive than compounds carrying a sulphonate-group. Further they do not over-strip, i.e., they do not degrease to leave an excessively dry or ‘squeaky’ feel the skin or hair. Accordingly, said surfactants are especially preferred for surfactant-assisted synthesis of nanoparticles, oil-recovery with surfactant flooding, foam boosters and laundry applications. In addition, they are particularly useful as surfactants in oil recovery, in assisted synthesis of nanoparticle, as foam boosters and additives in cleaning formulations for laundry applications. In particular, it was found that said compounds provide low interfacial tensions and low microemulsion viscosities.

In one embodiment of the present invention relates to a compound of the general formula (I), (II) and (III) preferably a compound of formula Ia, Ib, Ic, IIa, IIb, IIc, IIIa, IIIb, and IIIc, wherein R₅₀ or R₆₀ is selected from the group consisting of Z—SO₃⁻ and Z—OH, preferably Z—SO₃⁻, and —Z is preferably a linear or branched C₁ to C₁₀ alkyl, more preferably a linear C₁ to C₅ alkyl and most preferably a linear C₁ to C₃ alkyl. Most preferably said compound is selected from the group consisting of compound Ia, IIa and IIIa, wherein R₅₀ is Z—OH and R₆₀ is Z—SO₃⁻ and Z is preferably a linear or branched C₁ to C₁₀ alkyl, more preferably a linear C₁ to C₅ alkyl and most preferably a linear C₁ to C₃ alkyl. Said compounds are more labile than other surfactants, but they provide an excellent detergency and stable foamability. They are especially preferred as foaming agents and as detergents. Preferably, said compounds are used in shampoos as foaming agent or detergent. Said shampoos contain significant proportions of said compounds in an aqueous medium which has preferably about a neutral pH. The shampoo has most preferably a pH in the range of 6.5 to 7.5, more preferably 6.8 to 7.3. Such shampoos have excellent foaming properties and a good rinsability. Furthermore, the foams provide a good stability.

The compounds of the present invention, in particular compounds according to the general formula (Ia), (Ib), (Ic) and (V) can be used as surfactants, preferably as emulsifiers, foam stabilizers, wetting agents, emollients in cosmetic products, surfactants in food products, as anti-spattering agents for frying, as surfactant in pharmaceutical products, detergents or additives in cleaning products.

The present invention also encompasses a composition containing the compounds of the present invention and significant amounts of water. The inclusion of higher water amount is beneficial to incorporate more hydrophilic ingredients into the composition.

In one embodiment of the present invention the compounds are used as emulsifiers, foam stabilizers, wetting agents, emollients in cosmetic products, surfactants in food products, as anti-spattering agents for frying or as surfactant in pharmaceutical products.

The compounds of the present invention can be easily obtained by the following reaction steps:

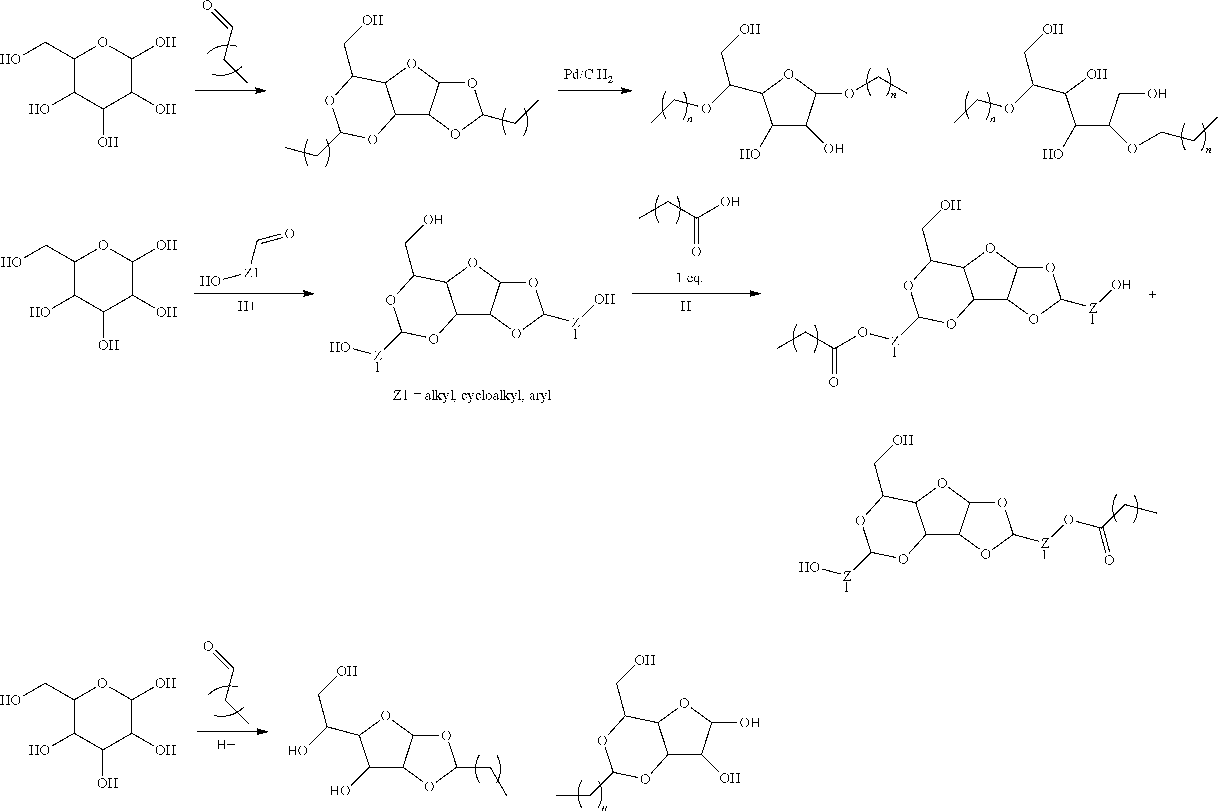

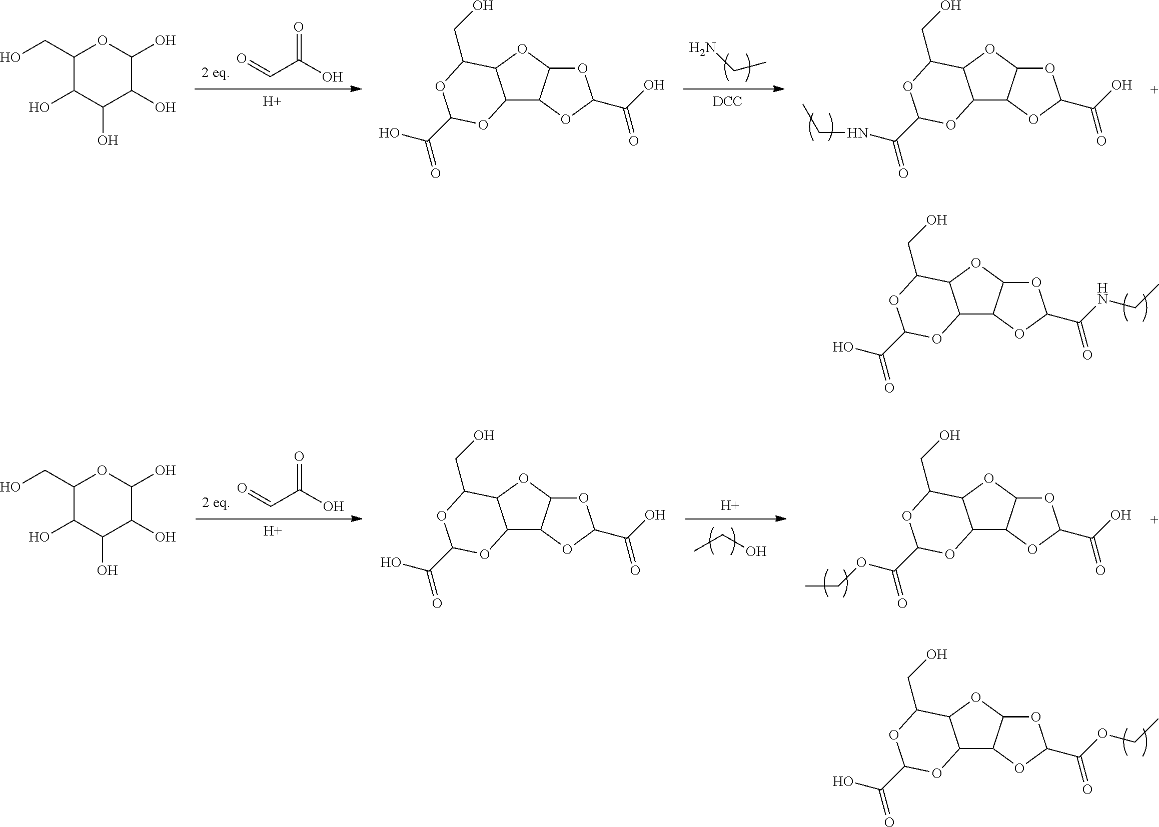

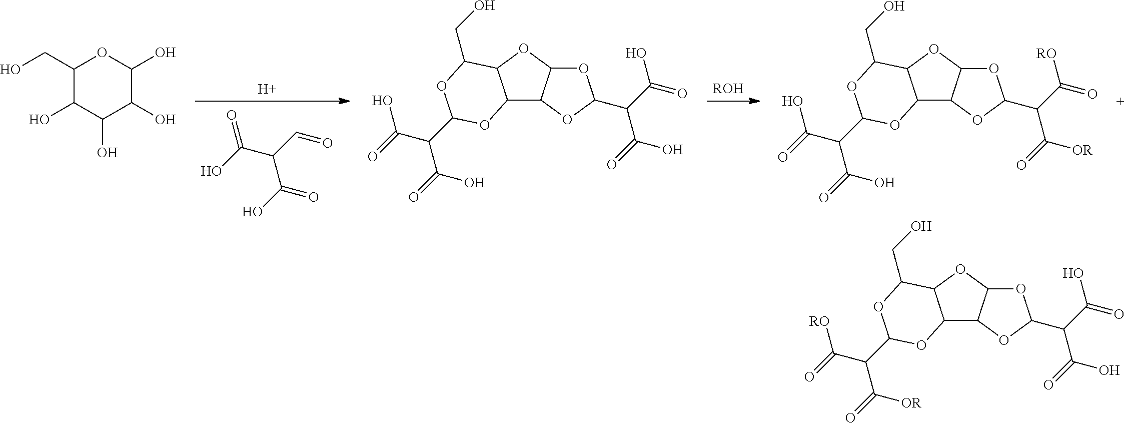

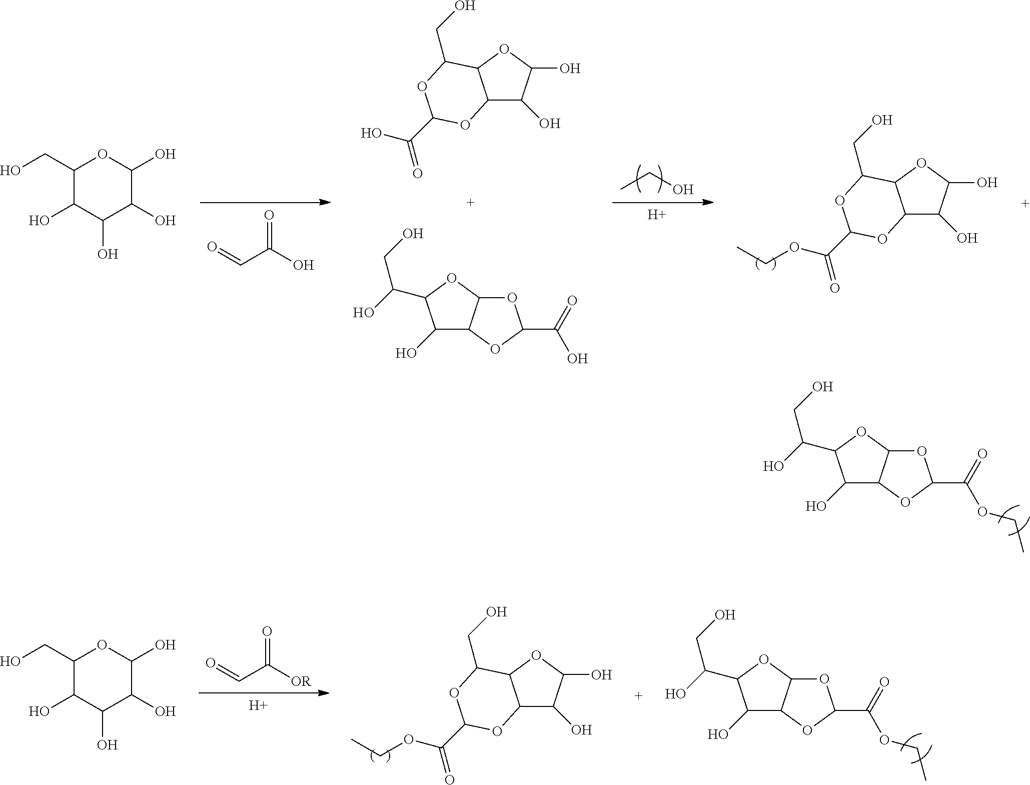

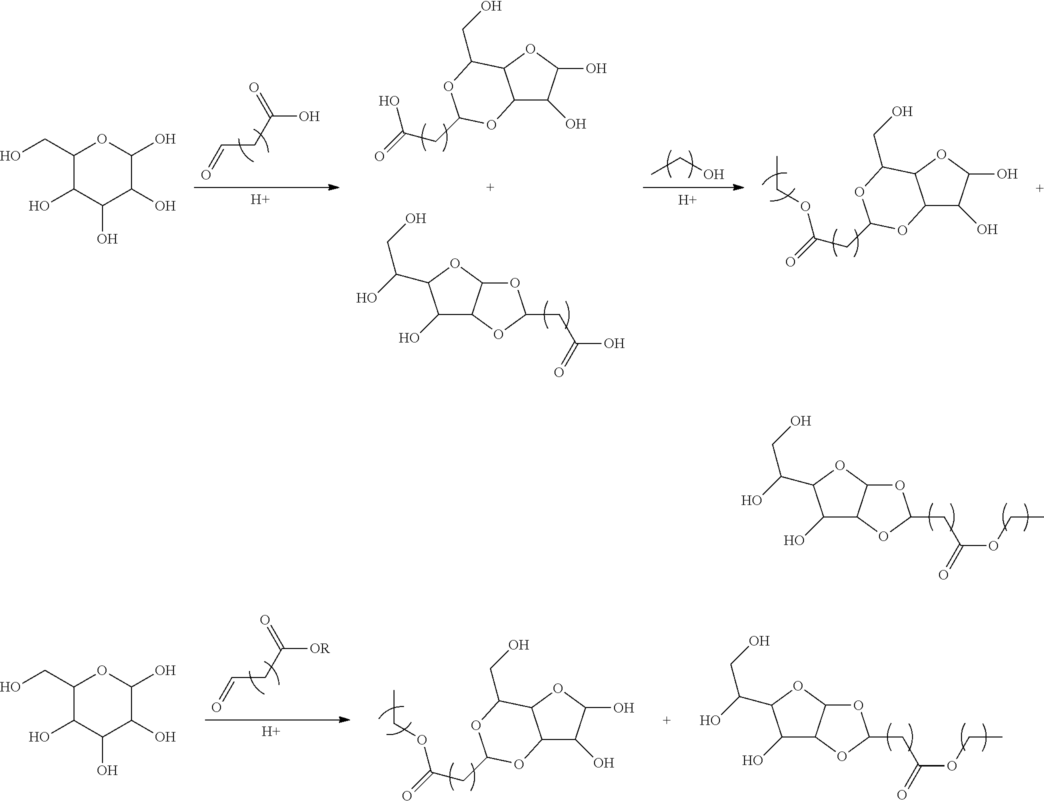

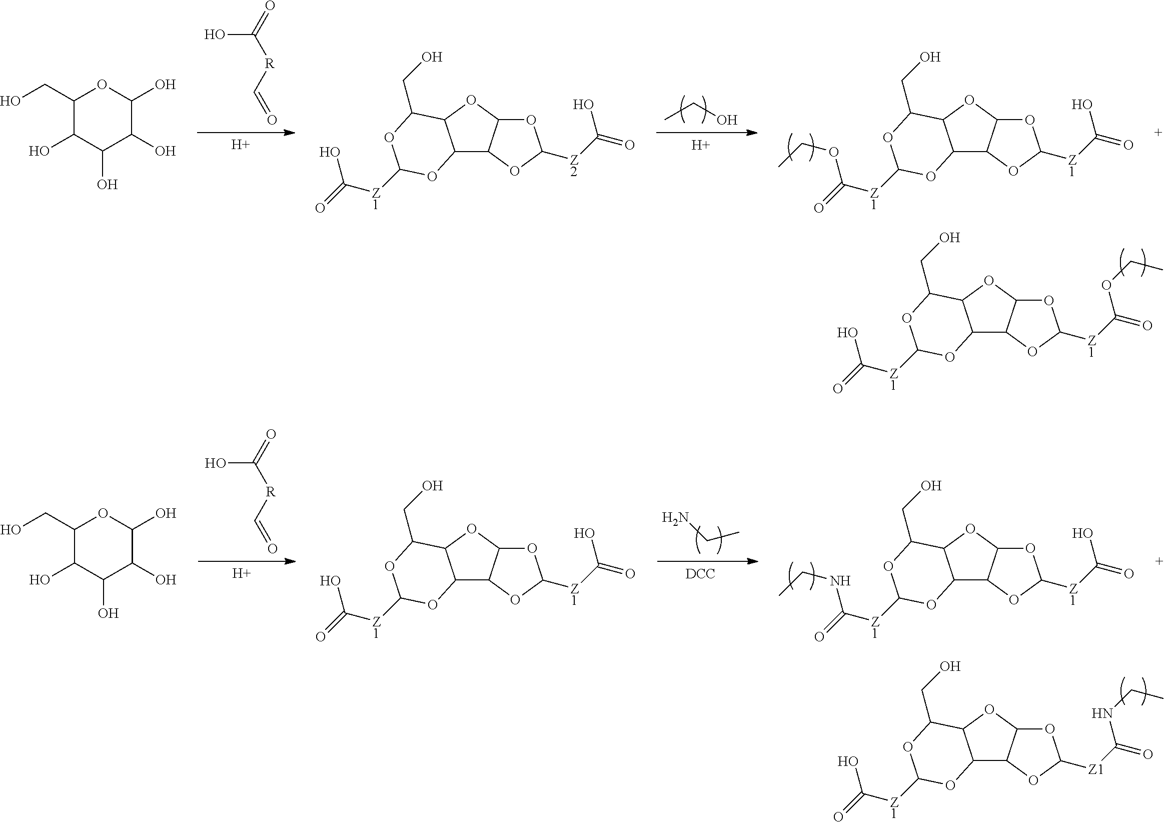

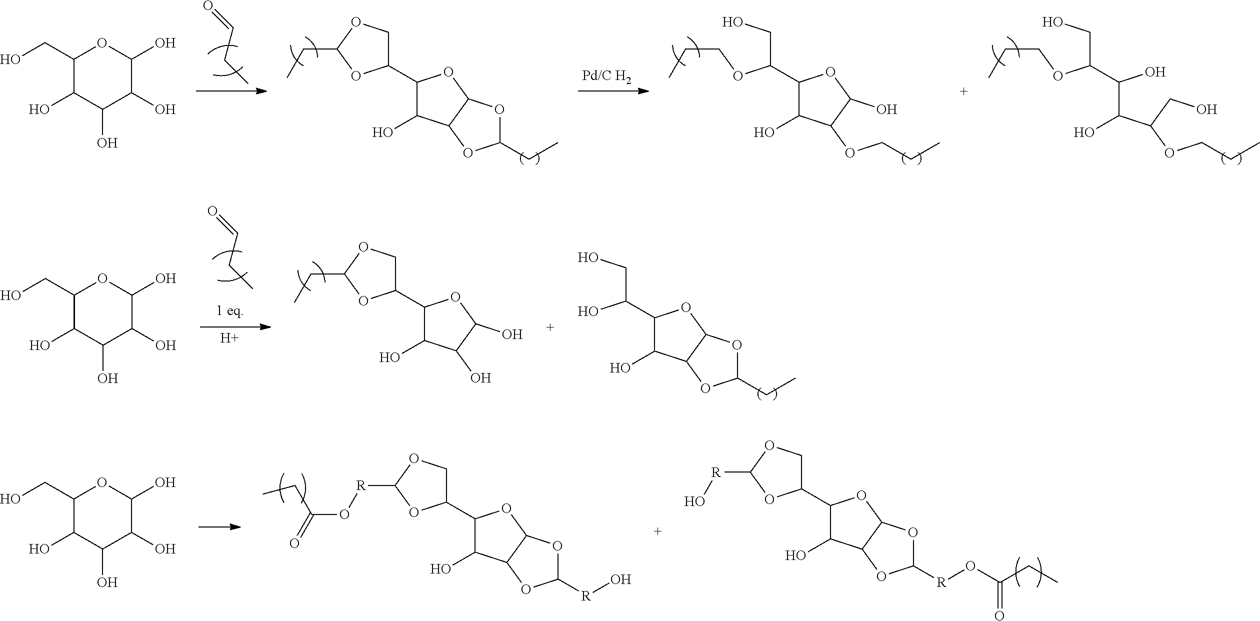

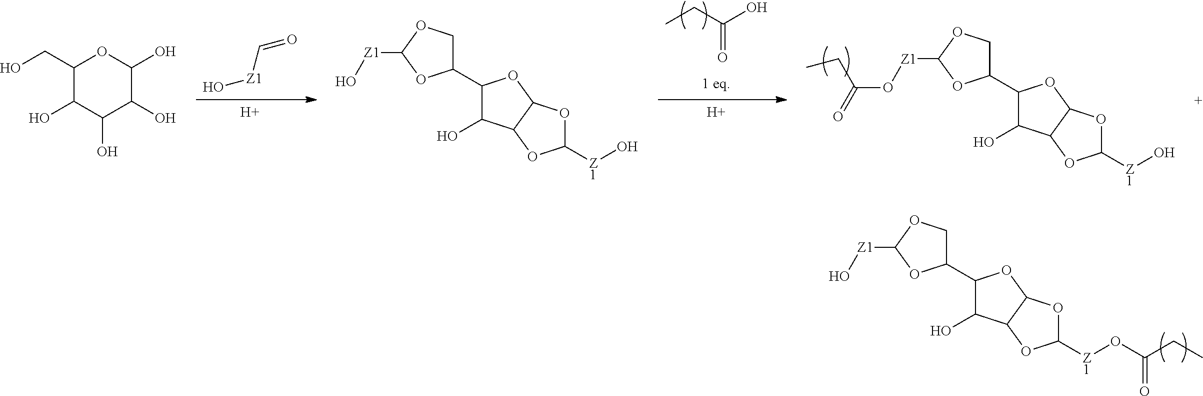

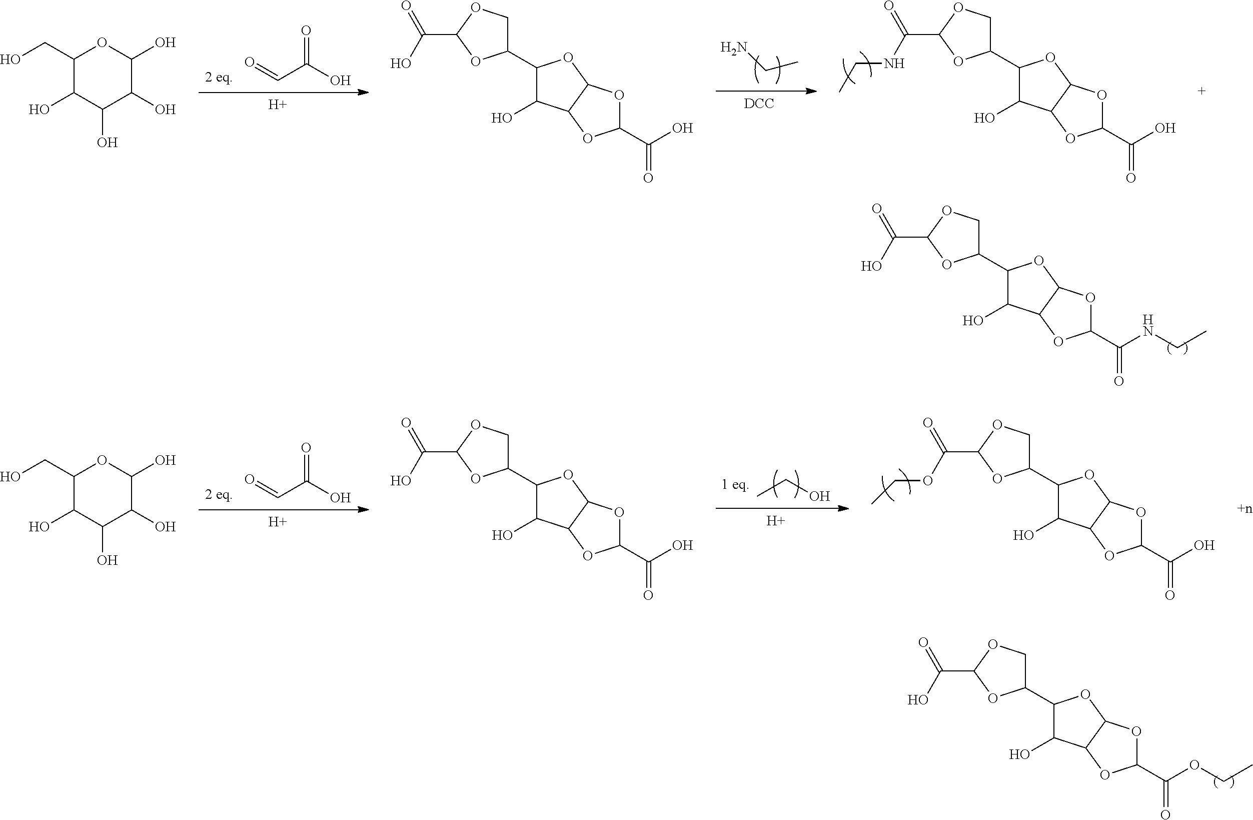

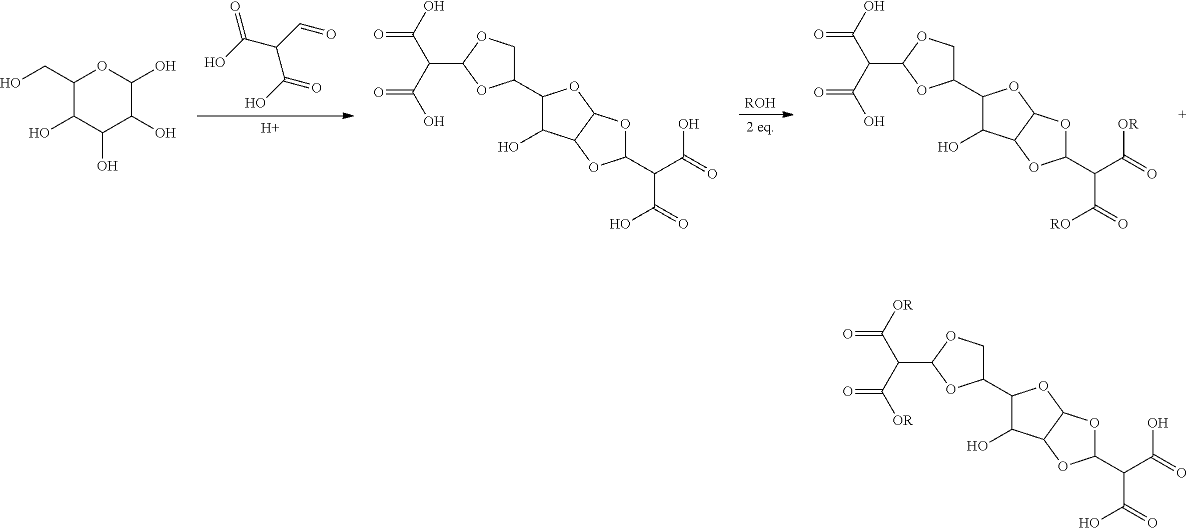

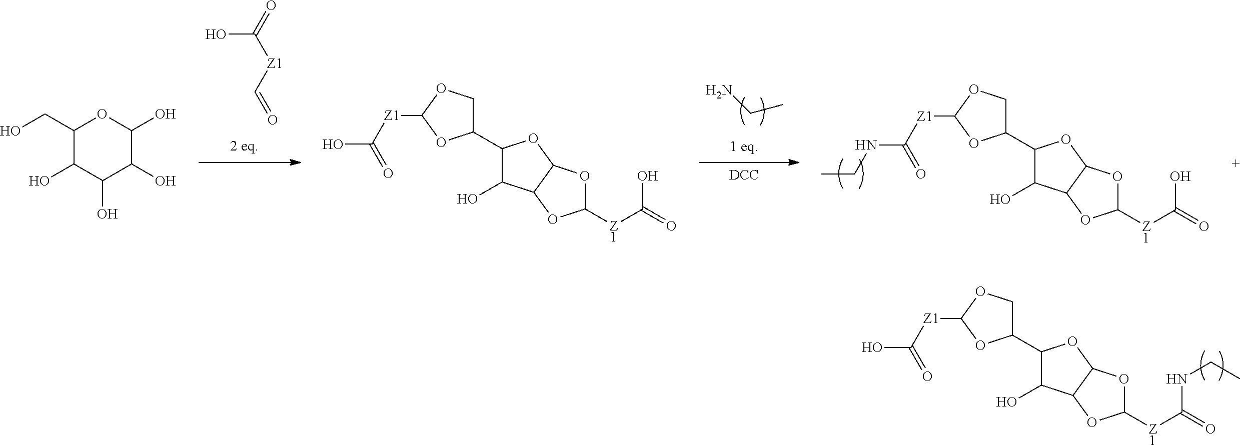

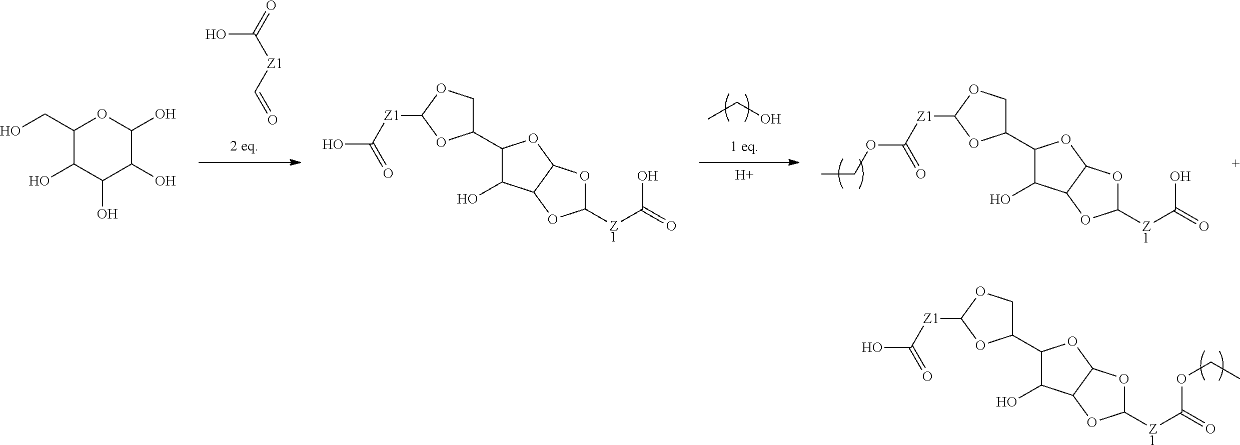

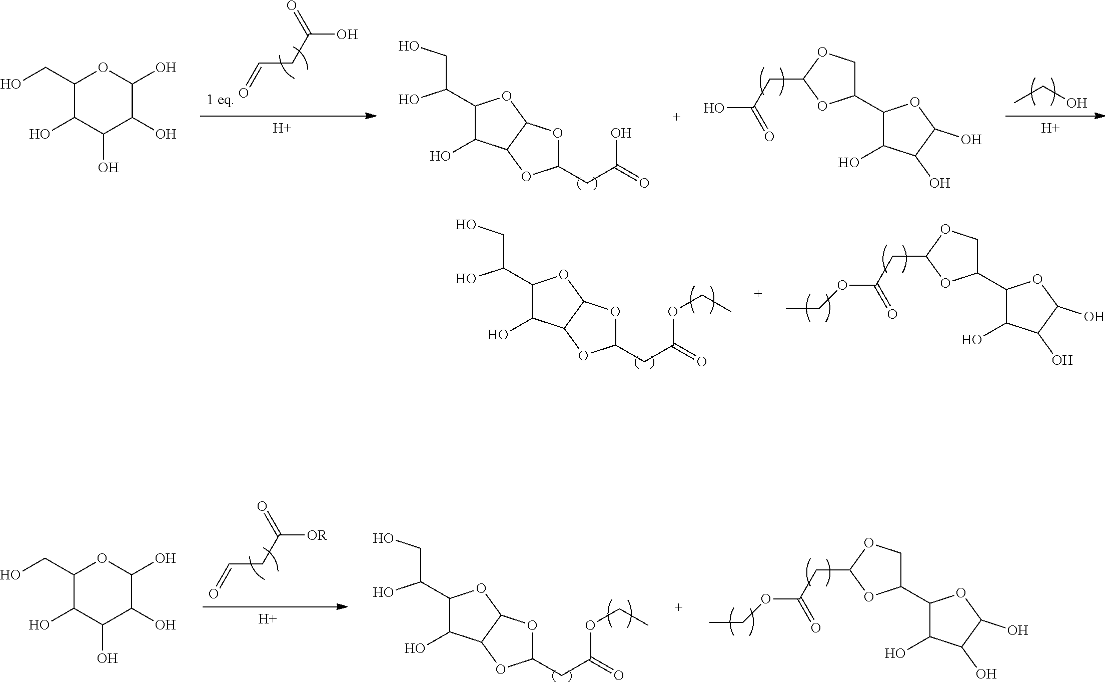

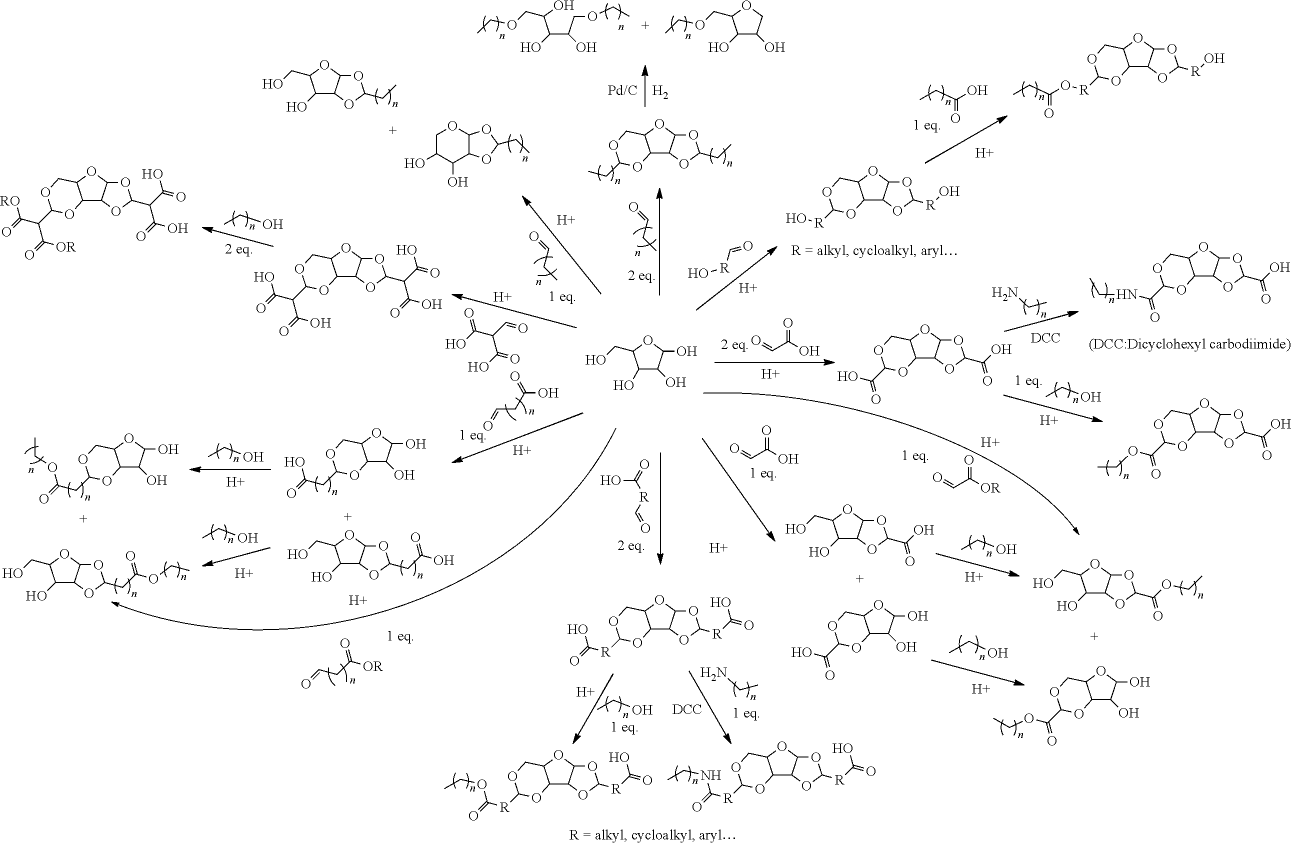

### DCC: Dicyclohexyl Carbodiimide

### EXAMPLES

### Synthesis of docosanol-substituted diglyoxylic acid xylose (2,3) and characterization

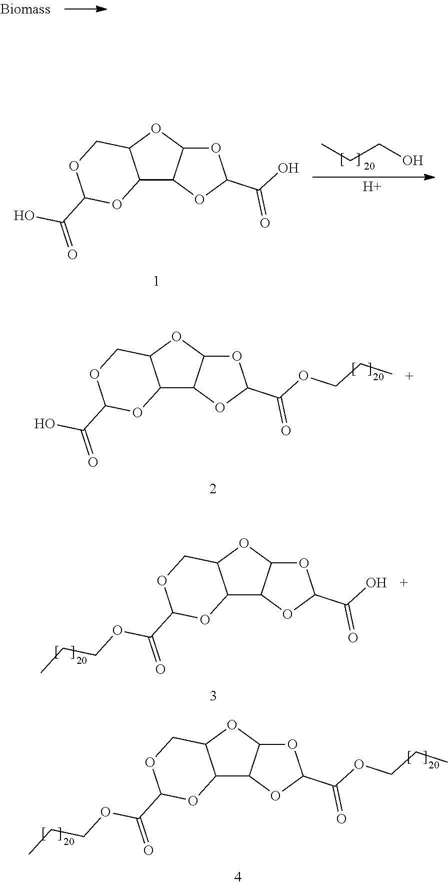

The synthesis of diglyoxylic acid-xylose (1) from xylose and biomass is known to the skilled person. Docosanol (0.56 g, 1.7 mmol) dissolved in 25 ml of chloroform and diglyoxylic acid-xylose (1) (0.5 g, 1.9 mmol) dissolved in 15 ml of 1,4-dioxane were mixed in 100 ml round-bottom flask. Sulfuric acid (98% pure, 200 mkl) was added to the reaction mixture and the reaction mixture was heated at 65° C. for 24 h to obtain slightly pink solution. The reaction mixture was analyzed by HPLC (C18 column, isopropanol/methanol 1:9 mobile phase) and TLC (hexane/ethyl acetate 1:8), and one-side protected product was identified (2,3 or their mixture).

### Synthesis of octanol-substituted diglyoxylic acid xylose (2′,3′) and characterization

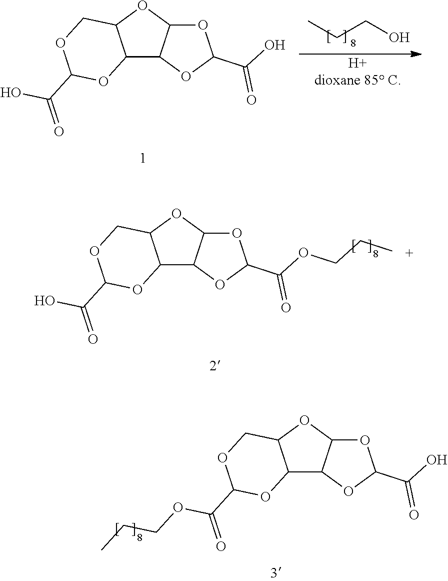

The synthesis of diglyoxylic acid-xylose (1) from xylose and biomass is known to the skilled person. 1-octanol (0.2 g, 1.7 mmol) and diglyoxylic acid-xylose (1) (0.5 g, 1.9 mmol) were mixed in 30 ml of 1,4-dioxane in 100 ml round-bottom flask. Sulfuric acid (98% pure, 200 mkl) was added to the reaction mixture and the reaction mixture was heated at 85° C. for 5 h. The reaction mixture was analyzed by HPLC (C18 column, isopropanol/methanol 1:9 mobile phase) and TLC (hexane/ethyl acetate 1:8), and one-side protected product was identified (2′,3′, or their mixture).

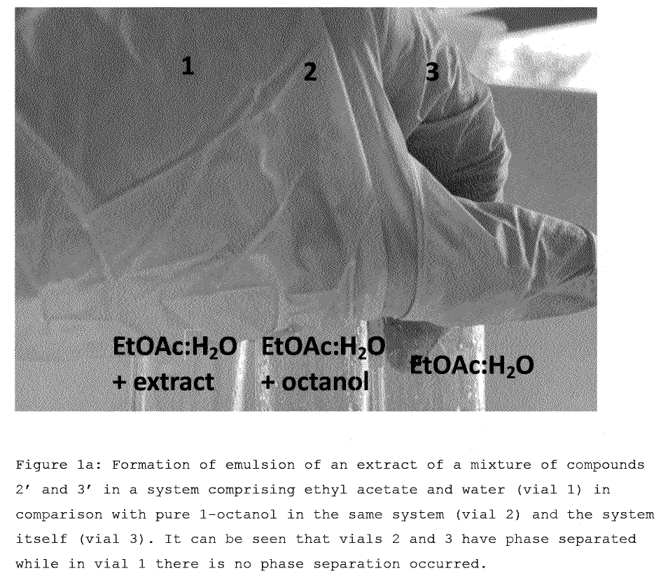

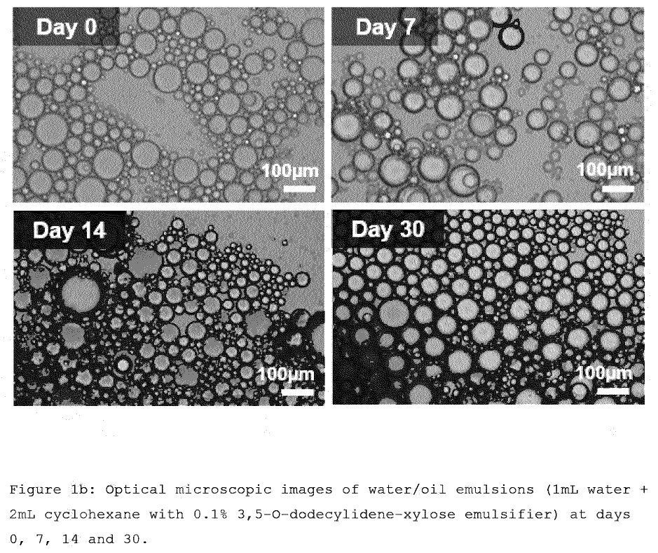

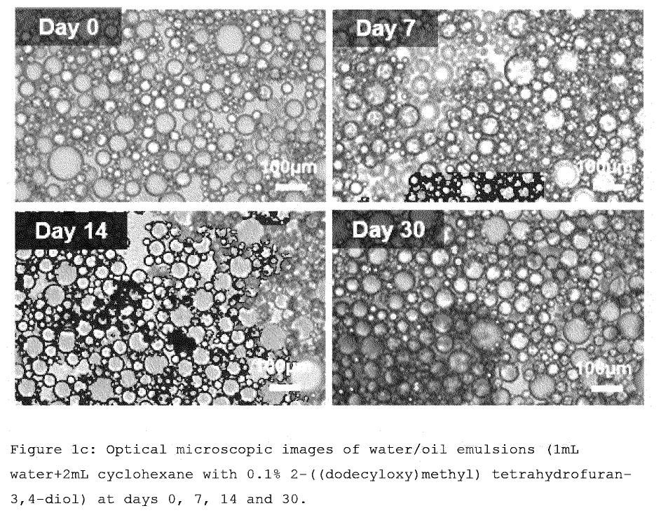

The mixture was neutralized until pH 7, and dissolved in a non-miscible system of ethyl acetate and water to do a quick emulsion test. FIG. 1a shows the formation of emulsion of an extract of a mixture of compounds 2′ and 3′ in a system comprising ethyl acetate and water (vial 1) in comparison with pure 1-octanol in the same system (vial 2) and the system itself (vial 3). It can be seen that vials 2 and 3 have phase separated while in vial 1 there is no phase separation occurred.

### Emulsion Stability Measurements

The emulsions were prepared by mixing 1 ml of water (with 1 mg/ml of Acian blue dye) and 2 ml cyclohexane containing 3,5-O-dodecylidene-xylose or 2-((dodecyloxy)methyl) tetrahydrofuran-3,4-diol) at a concentration of 0.1% and then mixed by vortex for 30 s.

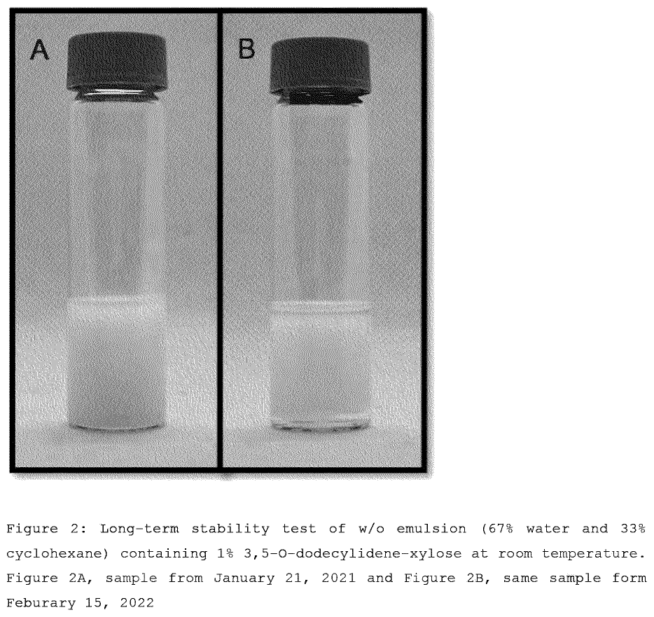

Emulsions were characterized using a bright-field microscope (Leitz Ergolux) during the storage after preparation. FIG. 1b and FIG. 1c show that the aqueous bubbles in oil phase can maintain stable for at least 30 days without obvious coalescence. Besides, a water/oil emulsion (67% water and 33% cyclohexane) containing 1% 3,5-O-dodecylidene-xylose was kept for about 1 year to observe its possible destablization. FIG. 2 shows a thin oil layer and a clarification layer was developed after 1 year, but most of the volume maintained the form of emulsion.

### Synthesis of 1,2-O-dodecylidene-xylose (6) and 3,5-O-dodecylidene-xylose (7) from xylose

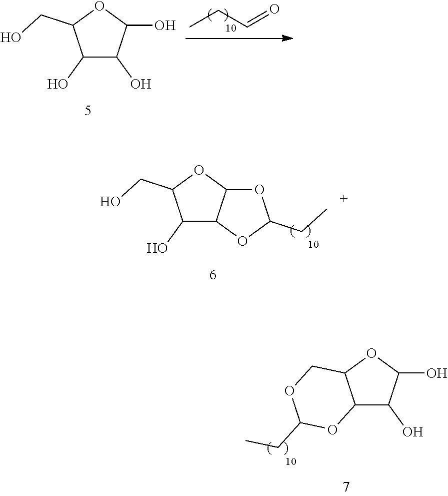

In a 1-neck round bottom flask, 1 molar equivalent of D-xylose was mixed with 0.9 molar equivalent dodecanal and 0.1 molar equivalents of sulfuric acid catalyst in dioxane. The reaction was conducted at 65° C. for 24 h. Then, the solution was neutralized with 1M NaOH solution until the pH value becomes around 7. The solution was concentrated on a rotavap with a bath temperature of 45° C. under reduced pressure (80 mbar). Then the residue viscous yellow oil was washed with brine solution and extracted with EtOAc. The organic phase was then evaporated on a rotavap and purified using column chromatography to obtain light yellow solid and yellowish oil. They were characterized by Heteronuclear Single Quantum Coherence Spectroscopy (HSQC) NMR.

### Synthesis of 1,2-O-carboxylidene-3,5-O-dodecylidene-xylose (9) from 3,5-O-dodecylidene-xylose (7)

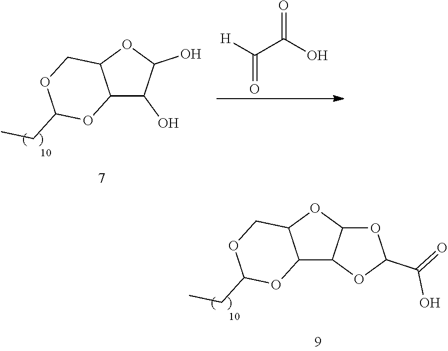

In a 1-neck round bottom flask, 1 molar equivalent of 3,5-O-dodecylidene-xylose (7) (MAX12) was mixed with 2 molar equivalent of glyoxylic acid monohydrate in dioxane. Amberlyst A15 was used as catalyst and molecular sieve was added to remove produced water. The reaction was conducted at 80° C. for 4 h. Then, the solution was filtered and concentrated on a rotary evaporator with a bath temperature of 45° C. under reduced pressure (80 mbar). Then the reaction mixture was purified using column chromatography (hexane-ethyl acetate with 1% acetic acid) to obtain 1,2-O-carboxylidene-3,5-O-dodecylidene-xylose (9) (GMAX) as a light yellow solid.

### Synthesis of sodium 1,2-O-carboxylate-3,5-O-dodecylidene-xylose (10) from 1,2-O-carboxylidene-3,5-O-dodecylidene-xylose (9)

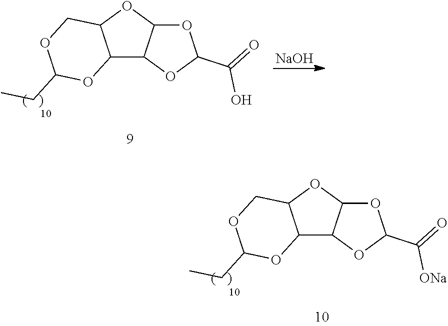

1 molar equivalent of 1,2-O-carboxylidene-3,5-O-dodecylidene-xylose (9) react with 1 molar equivalent of sodium hydroxide solution produce sodium 1,2-O-carboxylate-3,5-O-dodecylidene-xylose (10) with pH around 7.

### Synthesis of didodecylidene-xylose (8) from xylose

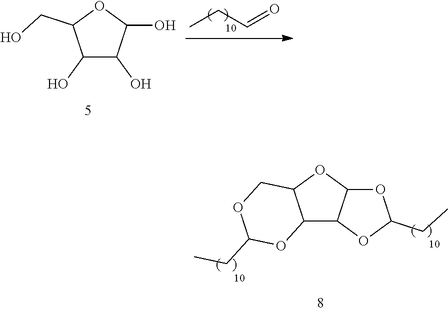

In a 1-neck round bottom flask, 1 molar equivalent of D-xylose was mixed with 2.05 molar equivalent dodecanal and 0.2 molar equivalents of sulfuric acid catalyst in dioxane. The reaction was conducted at 65° C. for 24 h. Then, the solution was neutralized with 1M NaOH solution until the pH value becomes around 7. The solution was concentrated on a rotavap with a bath temperature of 45° C. under reduced pressure (80 mbar). Then the resultant viscous pale-yellow oil was washed with brine solution and extracted with EtOAc. The organic phase was then evaporated on a rotavap and purified using flash chromatography. The product was collected and crystalized in hexane in the fridge to afford white didodecylidene-xylose crystals (8).

### Synthesis of 2-((dodecyloxy)methyl)tetrahydrofuran-3,4-diol

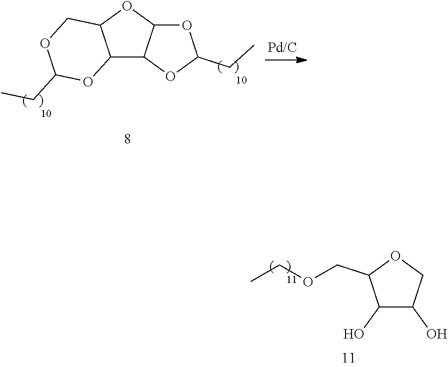 [SMILES: CCC1OCC2OC3OC(CC)OC3C2O1 ; CCOCC1OCC(O)C1O]

Didodecylidene-xylose (8) was dissolved in cyclopentyl methyl ether (CPME) and transferred to a 50-mL Parr reactor together with 10% Pd/C catalyst. The reactor was sealed and purged with hydrogen gas three times and hydrogen pressure was introduced (30 bar), and then heated to 135° C. for 15 hours with stirring. The reactor was depressurized after cooled to room temperature and the reaction mixture was filtered. The filtrate was evaporated on a rotary evaporator and the residue was purified by flash chromatography to give 2-((dodecyloxy)methyl)tetrahydrofuran-3,4-diol (11) and other dodecyl-xylose ethers and acetals.

### 3,5-O-(E)-dodec-2-en-1-ylidene-xylose (MAX12:1(2))

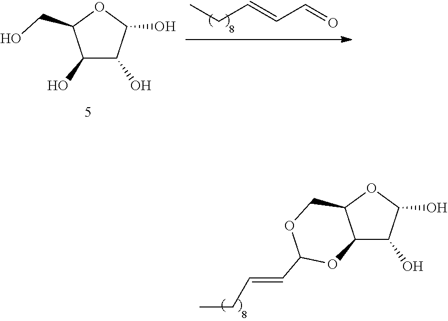

In a 1-neck round bottom flask, 1 molar equivalent of D-xylose (5) was mixed with 1.2 molar equivalent (E)-2-dodecenal and 0.02M of sulfuric acid catalyst in dioxane. In order to shift the equilibrium to the product, molecular sieve was added to remove produced water. The reaction was conducted at 45° C. for 15 h. Then, the solution was neutralized with 1M NaOH solution until the pH value becomes around 7. The solution was concentrated on a rotavap with a bath temperature of 45° C. under reduced pressure (80 mbar). Then the residue viscous yellow oil was washed with brine solution and extracted with EtOAc. The organic phase was then evaporated on a rotavap and purified using column chromatography to get two yellowish oil as the products (12). They were characterized by NMR and GCMS.

### Analytical Methods

### Terminology

In the context of the present invention the expression MAXn and DAXn refer to xylose compounds, wherein the term “n” defines the length of the variable linear alkyl group. For example the term MAX12 refers to 3,5-O-dodecylidene-xylose, the term MAX10 refers to 3,5-O-decylidene-xylose and the term MAX8 refers to 3,5-O-octylidene-xylose. On the other hand the expression DAXn refers to similar xylose targets, where the term “n” defines the length of both variable linear alkyl groups. For example the term DAX12 refers to didodecylidene-xylose, the term DAX10 refers to didecylidene-xylose and the term DAX8 refers to dioctylidene-xylose.

### NMR

All NMR spectra (¹H, ¹³C, HSQC) were acquired using a Bruker Avance III 400 MHz spectrometer using the standard pulse sequences from Bruker.

### GC-MS

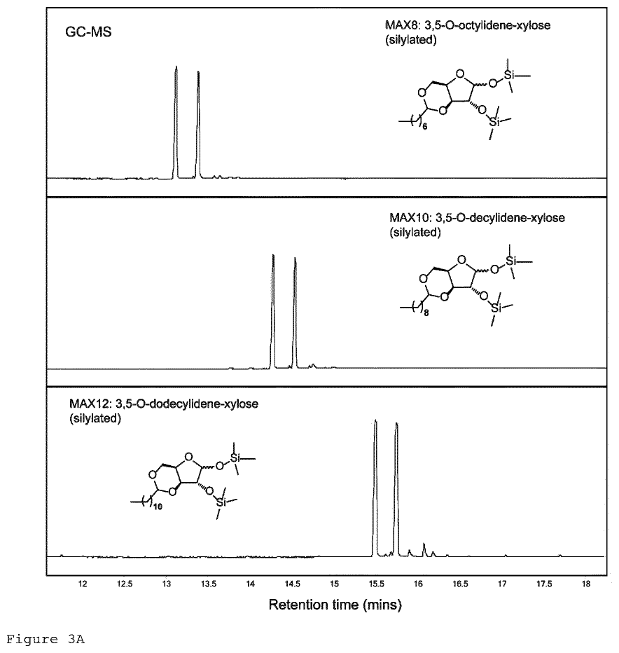

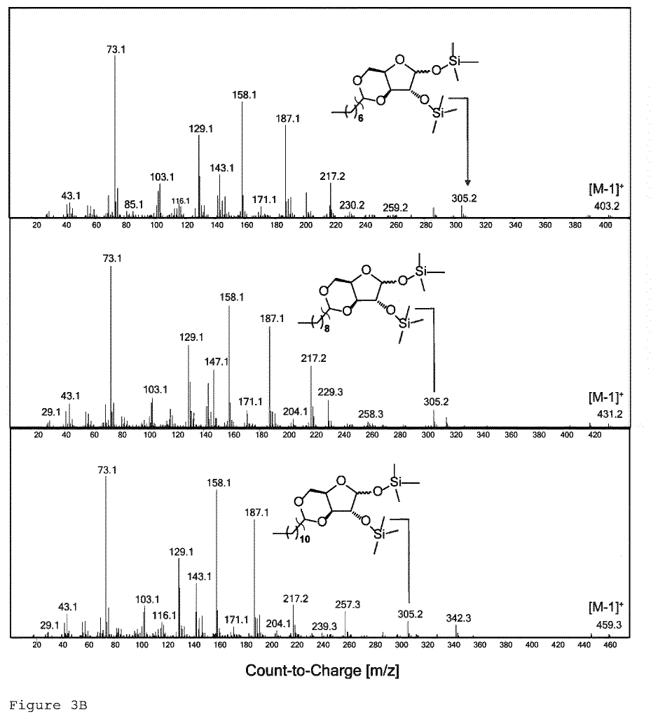

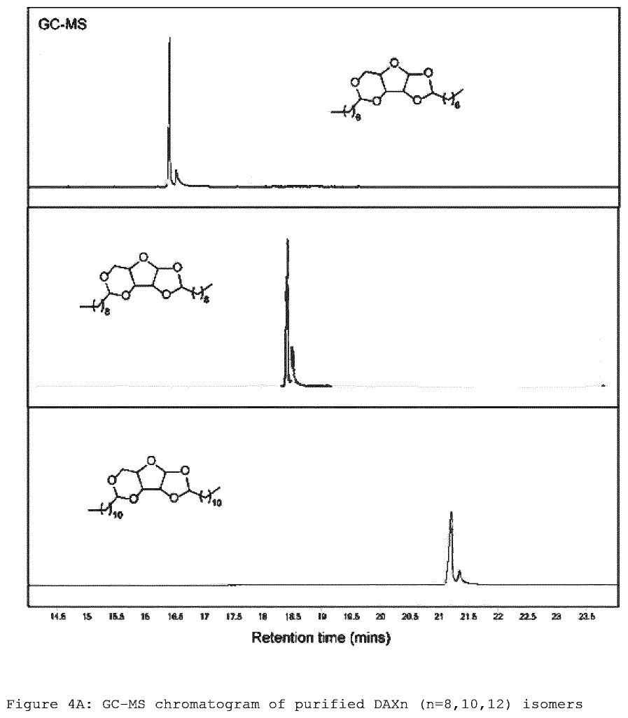

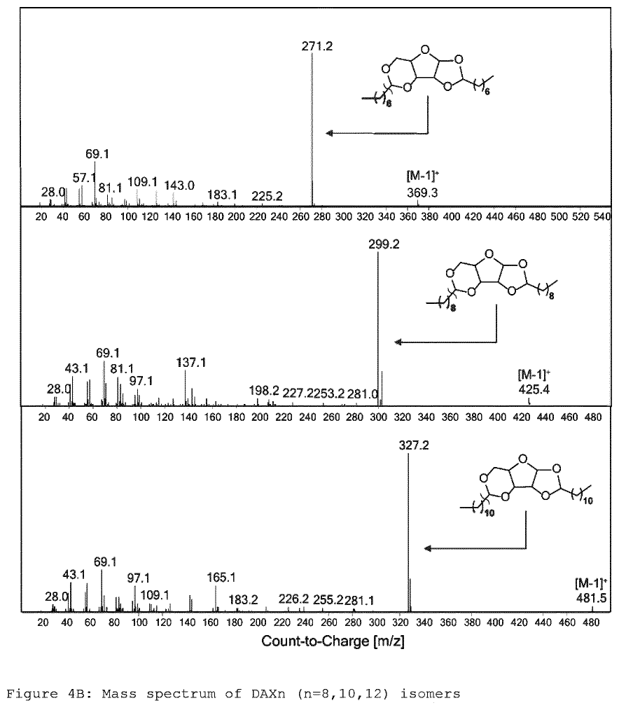

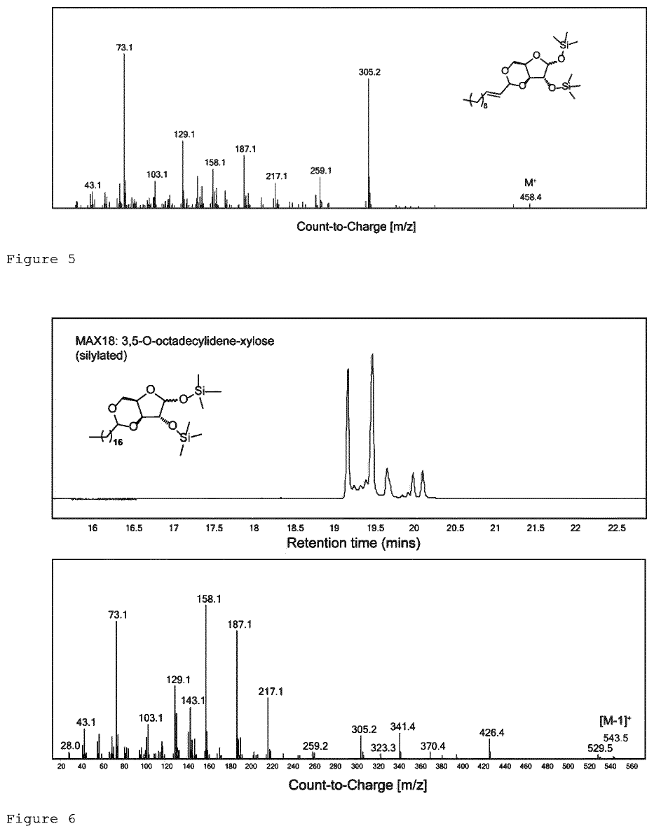

Gas chromatography-mass spectrometry spectra of 3,5-O-octylidene-xylose (MAX8), 3,5-O-decylidene-xylose (MAX10), 3,5-O-dodectylidene-xylose (MAX12) (all FIG. 3A), dioctylidene-xylose (DAX8), didectylidene-xylose (DAX10), didodectylidene-xylose (DAX12) (all FIG. 4A), 3,5-O-(E)-dodec-2-en-1-ylidene-xylose (MAX12:1(2) (FIG. 5) and 3,5-O-octadecylidene-xylose (MAX18) (FIG. 6) were obtained using an Agilent 7890B series GC equipped with a HP5-MS capillary column and an Agilent 5977A series Mass Spectroscopy detector (FIGS. 3B and 4B). A silylation derivatization was applied to all compounds mentioned above by adding 100 μL N-Methyl-N-(trimethylsilyl)-trifluoroacetamide (MSTFA) and 100 μL pyridine and kept under r.t. for 30 min before detection. The GC-MS method was performed as follows: The injection temperature was 300° C. 1 μL of sample was injected with an autosampler in split mode (split ratio: 25:1). The column was initially kept at 40° C. for 3 min, then was heated at a rate of 30° C. min⁻¹ to 100° C., followed by a heating rate of 40° C. min⁻¹ to 300° C. and held for 5 min.

### HPLC (pH 2 Aqueous-Phase Chromatography)

HPLC analyses for the accelerated aqueous decomposition of MAXn were performed using a HPX-87H Column (300 mm×7.8 mm; column temperature=60° C.) using pH 2 water as eluent (flow rate=0.6 ml·min⁻¹, Vᵢₙⱼ=20 μL) with 1260 Refractive Index Detector (RID) (G1362A)

### Characterization Data of Xylose Acetals

### 1,2-O-dodecylidene-α-D-xylofuranose (Ic)

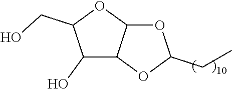

¹H NMR (400 MHz, CDCl₃) δ 5.96 (d, J=3.7 Hz, 1H), 5.17 (t, J=4.7 Hz, 0.52H), 4.92 (t, J=4.8 Hz, 0.48H), 4.49 (d, J=3.6 Hz, 1H), 4.42-4.31 (m, 2H), 4.16-3.93 (m, 4H), 1.16-1.42 (m, 18H), 0.86 (t, J=6.7 Hz, 6H).

¹³C NMR (101 MHz, CDCl₃) δ 106.91, 105.62, 104.65, 104.55, 86.43, 86.22, 81.47, 78.79, 77.09, 76.96, 61.33, 61.20, 34.72, 34.09, 32.03, 29.80, 29.76, 29.75, 29.73, 29.71, 29.63, 29.62, 29.56, 29.54, 29.48, 29.46, 23.85, 23.65, 22.80, 14.24.

HRMS (nanochip-ESI/LTQ-Orbitrap) m/z: [M+H]⁺ Calcd for C₁₇H₃₃O₅⁺317.2323; Found 317.2318.

### 1,2-O-carboxylidene-3,5-O-dodecylidene-xylose (Ia) (GMAX)

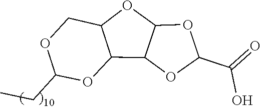

¹H NMR (400 MHz, CDCl₃) δ 6.22 (dd, J=33.6, 3.7 Hz, 1H), 5.48 (d, J=54.7 Hz, 1H), 4.69 (dd, J=57.0, 3.7 Hz, 1H), 4.46 (td, J=5.3, 3.1 Hz, 1H), 4.33-4.18 (m, 2H), 4.01 (q, J=1.8 Hz, 1H), 3.91 (ddd, J=13.5, 6.5, 2.0 Hz, 1H), 1.67-1.50 (m, 2H), 1.26 (d, J=4.3 Hz, 18H), 0.88 (t, J=6.8 Hz, 3H).

¹³C NMR (101 MHz, CDCl₃) δ 176.92, 171.60, 170.81, 106.66, 106.58, 100.56, 100.48, 100.19, 99.83, 85.97, 84.72, 77.96, 77.80, 73.47, 73.45, 67.19, 66.28, 65.99, 34.75, 34.72, 32.06, 29.78, 29.76, 29.67, 29.62, 29.49, 23.89, 22.83, 20.74, 14.26.

### 2-((dodecyloxy)methyl)tetrahydrofuran-3,4-diol (Va)

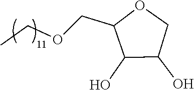

¹H NMR (400 MHz, CDCl₃) δ 4.27 (dt, J=3.9, 1.6 Hz, 1H), 4.24-4.17 (m, 2H), 4.16-4.09 (m, 1H), 3.91-3.78 (m, 2H), 3.74-3.67 (m, 1H), 3.58-3.42 (m, 2H), 1.63-1.53 (m, 2H), 1.34-1.22 (m, 18H), 10.87 (t, J=6.8 Hz, 3H).

¹³C NMR (101 MHz, CDCl₃) δ 79.39, 78.24, 78.16, 73.70, 72.62, 70.06, 32.04, 31.56, 29.78, 29.75, 29.72, 29.67, 29.61, 29.53, 26.13, 22.81, 14.24.

HRMS (ESI/QTOF) m/z: [M+Na]⁺ Calcd for C₁₇H₃₄NaO₄⁺325.23452; Found 325.23455.

### 3-O-dodecyl-1,2-O-dodecylidene-xylose (Ie)

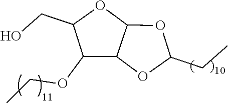

¹H NMR (400 MHz, CDCl₃) δ 5.96 (d, J=3.7 Hz, 1H), δ 5.17 (t, J=4.7 Hz, 1H), δ 4.49 (dd, J=3.7 Hz, 1H), δ 4.34-4.37 (m, 1H), 4.09-4.14 (m, 1H), 3.78-3.89 (m, 2H), 3.40-3.56 (m, 2H), 1.20-1.36 (m, 36H), 0.87 (t, J=7.08 Hz, 6H).

¹³C NMR (101 MHz, CDCl₃) δ 106.79, 104.68, 86.28, 80.59, 76.99, 72.72, 69.45, 34.77, 32.94, 32.05, 29.83, 29.80, 29.78, 29.76, 29.73, 29.67, 29.65, 29.58, 29.56, 29.53, 26.25, 23.70, 22.83, 14.25.

HRMS (ESI/QTOF) m/z: [M+Na]⁺ Calcd for C₂₉H₅₆NaO₅⁺507.4020; Found 507.4026.

### 5-O-dodecyl-1,2-O-dodecylidene-xylose (Id)

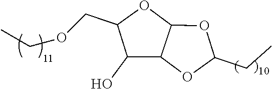

(b) ¹H NMR (400 MHz, CDCl₃) δ 5.94 (d, J=4.0 Hz, 1H), 4.92 (t, J=4.8 Hz, 1H), 4.39 (d, J=4.0 Hz, 1H), 4.31 (d, J=2.6 Hz, 1H), 4.18 (q, J=3.1 Hz, 1H), 4.00-3.87 (m, 2H), 3.68-3.43 (m, 2H), 1.72-1.57 (m, 2H), 1.51-1.61 (m, 2H), 1.33-1.23 (m, 36H), 0.87 (t, J=6.7 Hz, 6H).

¹³C NMR (101 MHz, CDCl₃) δ 105.40, 104.58, 86.02, 77.97, 76.88, 72.85, 69.29, 34.12, 32.94, 32.06, 29.81, 29.79, 29.77, 29.75, 29.73, 29.67, 29.64, 29.58, 29.55, 29.53, 26.09, 23.90, 22.83, 14.26.

HRMS (ESI/QTOF) m/z: [M+Ag]⁺ Calcd for C₂₉H₅₆AgO₅⁺591.3173; Found 591.3180.

### Didodecylidene-xylose (DAX12) (Ia)

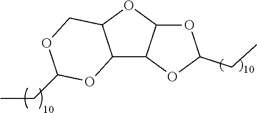

¹H NMR (400 MHz, CDCl₃) δ 6.01 (d, J=3.8 Hz, 1H), 4.94 (t, J=4.8 Hz, 1H), 4.51-4.39 (m, 2H), 4.32-4.22 (m, 1H), 4.20 (d, J=2.1 Hz, 1H), 4.02-3.98 (m, 1H), 3.96-3.88 (m, 1H), 1.72-1.55 (m, 4H), 1.43-1.17 (m, 36H), 0.87 (t, J=6.98 Hz, 6H).

¹³C NMR (101 MHz, CDCl₃) δ 105.48, 105.34, 100.57, 84.42, 78.48, 72.55, 66.22, 34.81, 34.77, 32.06, 32.05, 29.76, 29.68, 29.64, 29.62, 29.58, 29.51, 29.48, 29.39, 29.32, 29.21, 24.85, 23.97, 23.92, 23.80, 22.83, 14.26.

### Didecylidene-xylose (DAX10) (Ia)

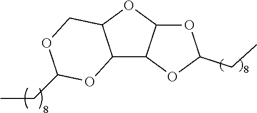

¹H NMR (400 MHz, CDCl₃) δ 5.99 (d, J=4.0 Hz, 1H), 4.94 (t, J=4.8 Hz, 1H), 4.50-4.39 (m, 1H), 4.33-4.23 (m, 2H), 4.20 (d, J=2.2 Hz, 1H), 3.96-3.82 (m, 2H), 1.72-1.52 (m, 4H), 1.44-1.22 (m, 28H), 0.87 (t, J=6.8 Hz, 6H).

¹³C NMR (101 MHz, CDCl₃) δ 107.28, 105.45, 100.57, 84.41, 78.49, 75.09, 72.55, 66.22, 34.77, 34.07, 32.02, 32.00, 29.63, 29.62, 29.60, 29.58, 29.55, 29.50, 29.43, 29.42, 23.96, 23.92, 23.80, 23.66, 22.81, 14.25.

### Dioctylidene-xylose (DAX8) (Ia)

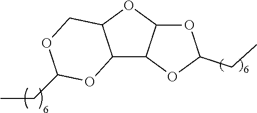

¹H NMR (400 MHz, CDCl₃) δ 5.99 (d, J=4.0 Hz, 1H), 4.94 (t, J=4.8 Hz, 1H), 4.52-4.39 (m, 2H), 4.33-4.23 (m, 1H), 4.20 (d, J=2.2 Hz, 1H), 4.04-3.98 (m, 1H), 3.96-3.82 (m, 1H), 1.72-1.52 (m, 4H), 1.45-1.14 (m, 20H), 0.90-0.82 (m, 6H).

¹³C NMR (101 MHz, CDCl₃) δ 107.28, 105.48, 100.57, 84.41, 78.49, 75.09, 72.55, 66.40, 34.77, 34.07, 31.87, 31.85, 29.54, 29.51, 29.46, 29.29, 29.28, 23.96, 23.92, 23.80, 23.66, 22.77, 22.75, 14.22.

### 3,5-O-(E)-dodec-2-en-1-ylidene-xylose (MAX12:1(2)) (Ib)

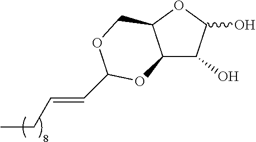

¹H NMR (400 MHz, CDCl₃) δ 7.12-6.69 (m, 1H), 6.20-5.78 (m, 1H), 5.66 (dd, J=8.5, 3.8 Hz, 1H), 5.38 (ddt, J=5.7, 2.0, 1.1 Hz, 1H), 4.82 (dd, J=4.2, 3.3 Hz, 10H), 4.64-3.62 (m, 5H), 2.36-1.84 (m, 2H), 1.20 (d, J=12.3 Hz, 14H), 0.81 (t, J=6.6 Hz, 3H).

¹³C NMR (101 MHz, CDCl₃) δ 159.34, 136.95, 136.85, 132.93, 125.80, 125.43, 124.62, 104.29, 99.30, 99.14, 97.74, 92.71, 80.52, 79.35, 79.12, 77.34, 77.23, 77.03, 76.71, 75.30, 73.60, 71.56, 66.85, 61.76, 32.76, 32.06, 32.04, 31.89, 31.85, 29.51, 29.50, 29.46, 29.44, 29.35, 29.31, 29.28, 29.26, 29.22, 29.18, 29.14, 28.57, 27.84, 22.67, 22.66, 14.11.

### 3,5-O-dodecylidene-xylose (MAX12) (Ib)

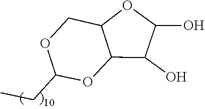

¹H (400 MHz, CDCl₃) δ 5.69 (d, J=3.8 Hz, 0.6H), 5.17 (s, 0.4H), 4.51-4.40 (m, 1H), 4.25-4.16 (m, 2H), 4.16-4.05 (m, 2H), 4.00-3.78 (m, 1H), 1.65-1.53 (m, 2H), 1.41-1.21 (m, 18H), 0.87 (t, J=6.7 Hz, 3H).

¹³C NMR (101 MHz, CDCl₃) δ 104.32, 100.52, 80.46, 73.94, 71.85, 67.40, 34.94, 34.83, 29.69, 29.64, 29.55, 29.47, 23.69, 22.80, 14.24.

HRMS (Sicrit plasma/LTQ-Orbitrap) m/z: [M+H−10-1]⁺ Calcd for C₁₋₇H₃₁O₄+299.2217; Found 299.2214.

### 3,5-O-octadecylidene-xylose (MAX18) (Ib)

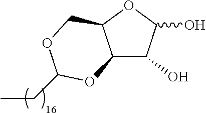

¹H NMR (400 MHz, CDCl₃) δ 5.65 (d, J=3.7 Hz, 0.6H), 5.11 (s, 0.4H), 4.25-4.16 (m, 2H), 4.16-4.05 (m, 2H), 4.00-3.78 (m, 1H), 1.65-1.53 (m, 2H), 1.42-1.01 (m, 30H), 0.81 (t, J=6.7 Hz, 3H).

¹³C NMR (101 MHz, CDCl₃) δ 104.32, 100.52, 80.46, 73.94, 71.85, 67.40, 34.94, 34.83, 29.69, 29.64, 29.55, 29.47, 23.69, 22.80, 14.24.

### Interfacial Tension Measurements

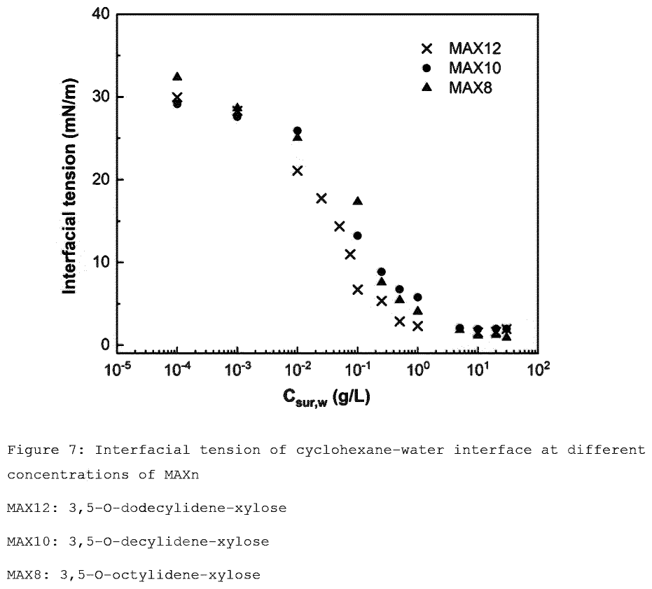

To test the amphiphilic properties of each molecule, we measured the water/oil interfacial tension using pendant drop test. In a typical test, we inject the organic solution with surfactants slowly into the water phase with a bent needle with 1 mm diameter. The interfacial tension is calculated by the force balance with the buoyancy force. The critical micelle concentration (CMC) can be obtained by checking the critical point of interfacial tension-concentration graph. FIG. 7 shows that MAX12 has lowest CMC (0.35 mg/mL) among tail length between 8-12. The CMC of MAX10 and MAX8 are around 2.5 mg/mL.

FIG. 8 shows that the CMC of 2-((dodecyloxy)methyl)tetrahydrofuran-3,4-diol (e) is around 0.5 g/L, and it can reduce the interfacial tension (cyclohexane/water) to a plateau value about 1.0 mN/m. 1,2-O-dodecylidene-xylose (d) has a CMC around 1 g/L and induce a decrease of the interfacial tension (cyclohexane/water) to a plateau value about 2.7 mN/m.

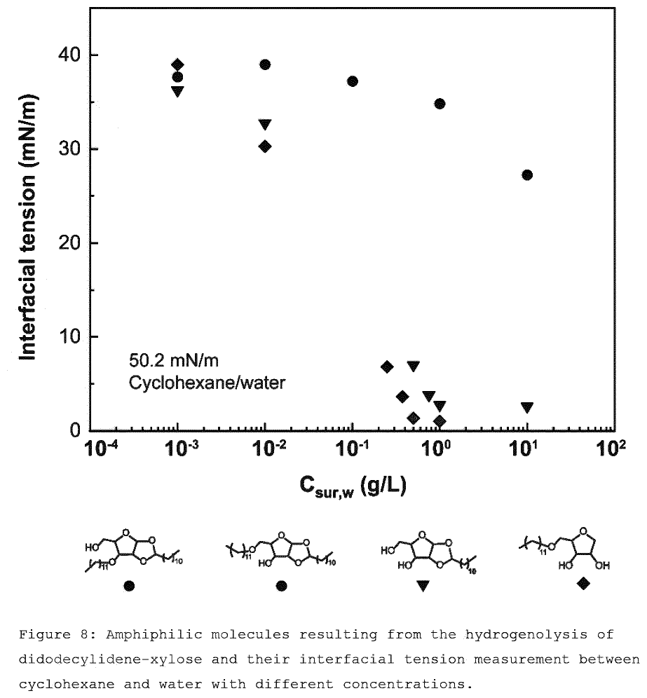

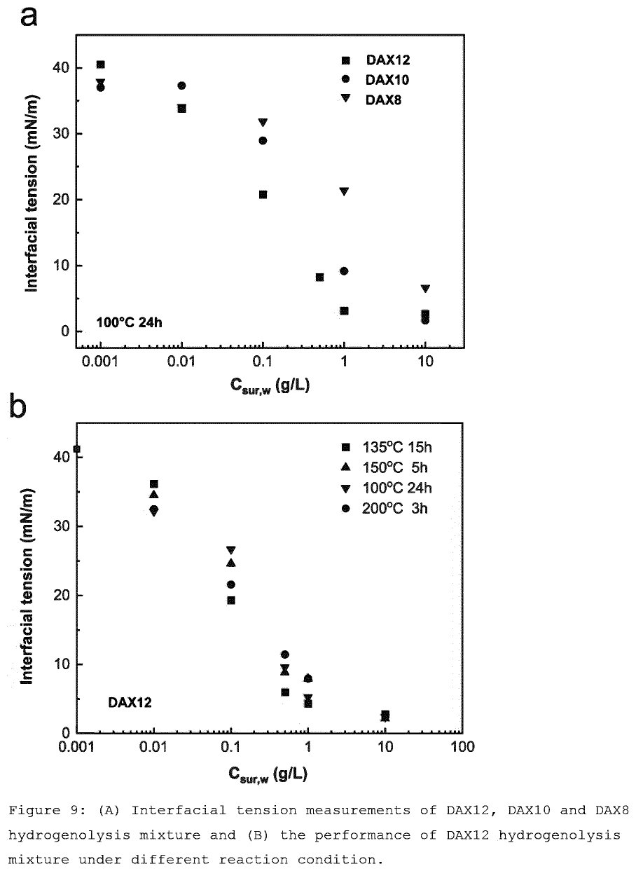

The reaction mixture without purification also has amphiphilic properties, which vary depending on the alkyl chain length shown by FIG. 9a. Among the length from C₈ to C₁₋₂, DAXn reaction mixture demonstrates the best ability of reducing the interfacial tension to a plateau value about 3 mN/m (cyclohexane/water). And the amphiplic properties of reaction mixture show differences under different hydrogenolysis conditions. For example, 135° C. for 15 h lowered the interfacial tension (cyclohexane/water) to 6 mN/m at 0.5 g/L, while 200° C. for 3 h lowered the interfacial tension(cyclohexane/water) to 11.5 mN/m at 0.5 g/L shown in FIG. 9b.

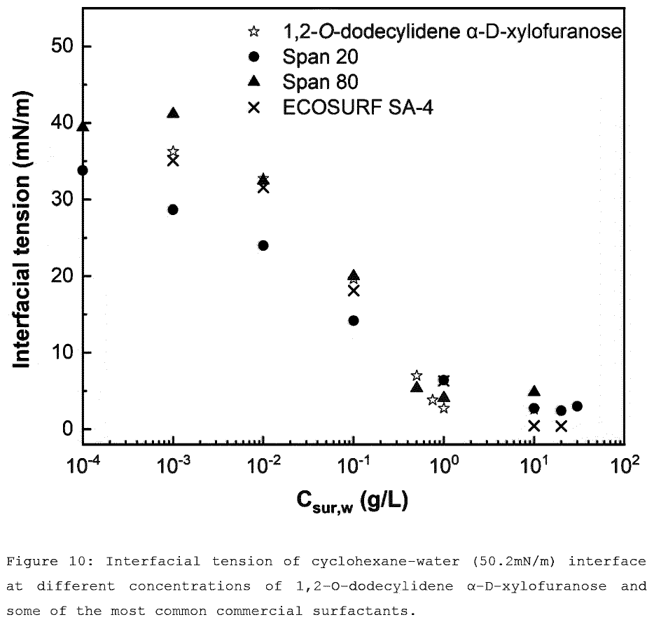

FIG. 10 shows the interfacial tension measurements of cyclohexane-water (50.2 mN/m) interface at different concentrations of 1,2-O-dodecylidene α-D-xylofuranose (Ic) and some of the most common commercial surfactants such as Span 20, Span 80 and ECOSURF SA-4.

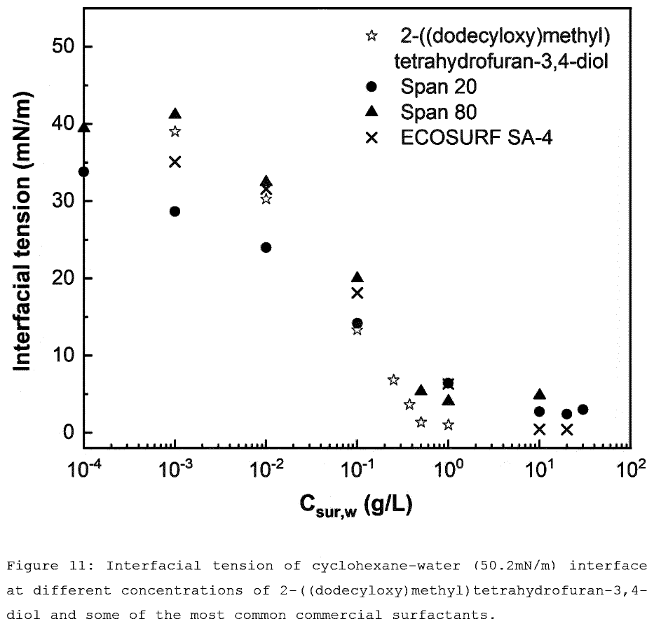

FIG. 11 shows the interfacial tension measurements of cyclohexane-water (50.2 mN/m) interface at different concentrations of 2-((dodecyloxy)methyl)tetrahydrofuran-3,4-diol (Va) and some of the most common commercial surfactants such as Span 20, Span 80 and ECOSURF SA-4.

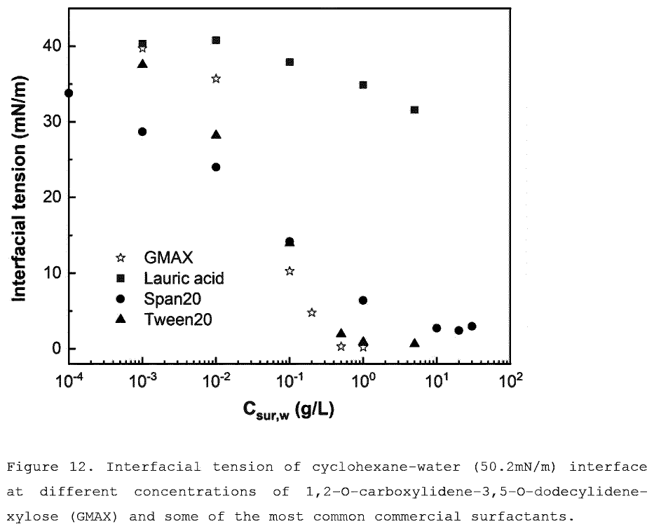

FIG. 12 shows the interfacial tension measurements of cyclohexane-water (50.2 mN/m) interface at different concentrations of 1,2-O-carboxylidene-3,5-O-dodecylidene-xylose (GMAX) (Ia) and some of the most common commercial surfactants such as Span 20, Span 80 and ECOSURF SA-4.

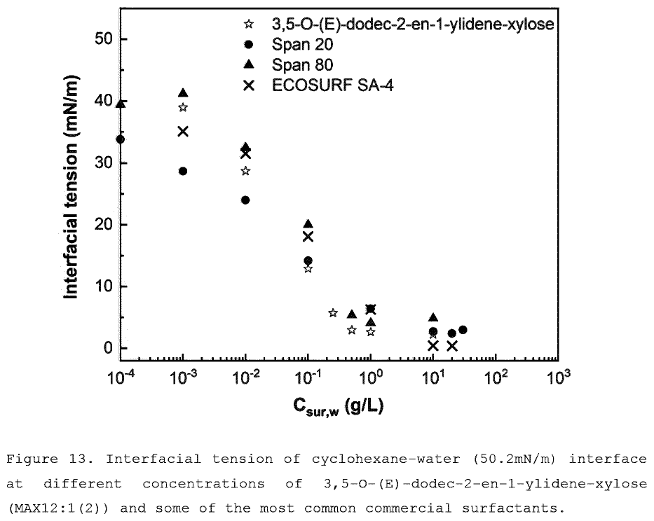

FIG. 13 shows the interfacial tension measurements of cyclohexane-water (50.2 mN/m) interface at different concentrations of 3,5-O-(E)-dodec-2-en-1-ylidene-xylose (MAX12:1(2)) (Ib) and some of the most common commercial surfactants such as Span 20, Span 80 and ECOSURF SA-4.

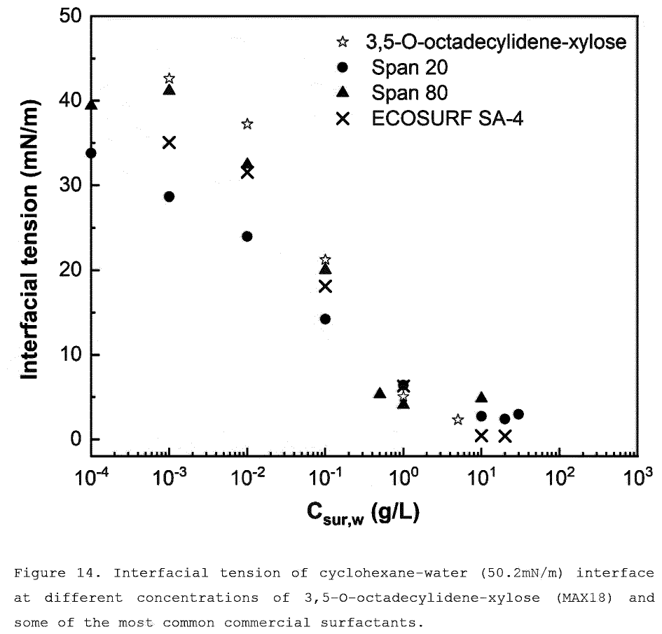

FIG. 14 shows the interfacial tension measurements of cyclohexane-water (50.2 mN/m) interface at different concentrations of 3,5-O-octadecylidene-xylose (MAX18) (Ib) and some of the most common commercial surfactants such as Span 20, Span 80 and ECOSURF SA-4.

### Surface Tension Measurement

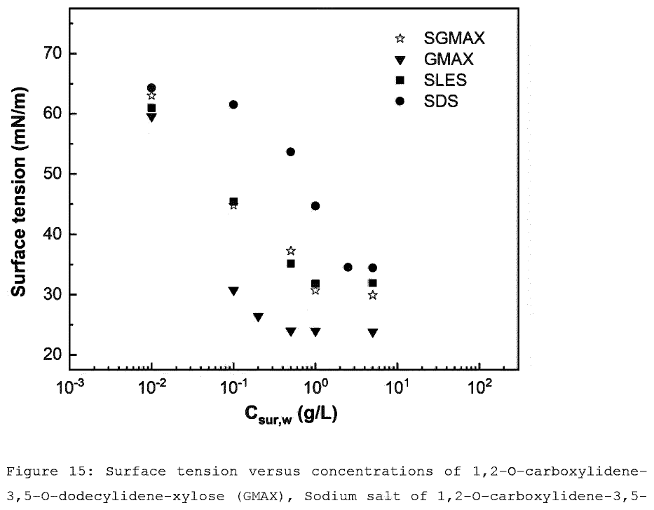

Amphiphilic properties of 1,2-O-carboxylidene-3,5-O-dodecylidene-xylose (GMAX) and sodium 1,2-O-carboxylate-3,5-O-dodecylidene-xylose (SGMAX), the ability of reducing the surface tension of the water, was measured using the pendant drop test. Prepare a series of different concentration surfactant aqueous solutions and load in a 1 mL syringe and install onto a Kruss SDA 30 drop shape analyzer. In a typical test, we slowly make a pendant drop of the surfactant aqueous solution and calculate the surface tension of water by analyzing the shape of the pendant drop (Young-Laplace equation) using the Kruss Advance software (v.1.6.2.0). The critical micelle concentration (CMC) is obtained by checking the point where the plateau starts to form in the surface tension-concentration graph (FIG. 15).

### Accelerated Aqueous Decomposition Test

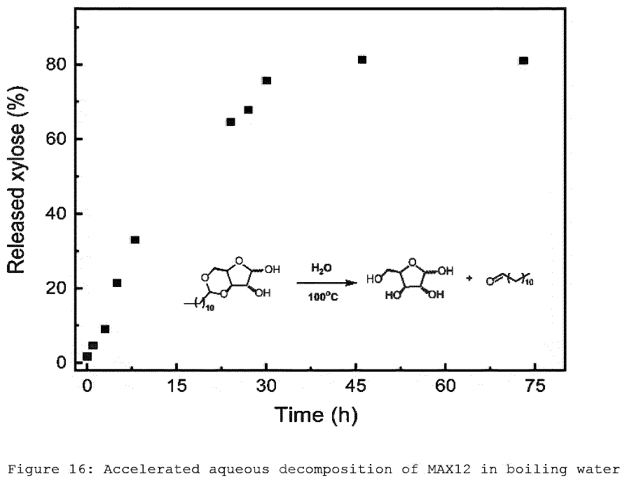

To gain insight into the degradation products of 1,2-O-dodecylidene-xylose (MAX12) in water, an accelerating aging test was performed by boiling it in water. Samples were taken at various time points and analyzed by HPLC (pH2 aqueous-phase chromatography, HPX-87H Column (300 mm×7.8 mm; 125-0140), 1260 Refractive Index Detector (RID) (G1362A), flow rate=0.6 ml·min⁻¹, Vᵢₙⱼ=20 μL, column temperature=60° C.). It shows that MAX12 can be cleaved into xylose and fatty aldehyde in boiling water in 2 days. And to our knowledge, fatty aldehydes can be oxidized into fatty acids catalyzed by the aldehyde dehydrogenase enzyme. Xylose and fatty acids are readily biodegradable. The result shows that the MAX12 can be easily decomposed and degraded after use (FIG. 16).

1. A compound of the general formula (Ia), (Ib) or (Ic) 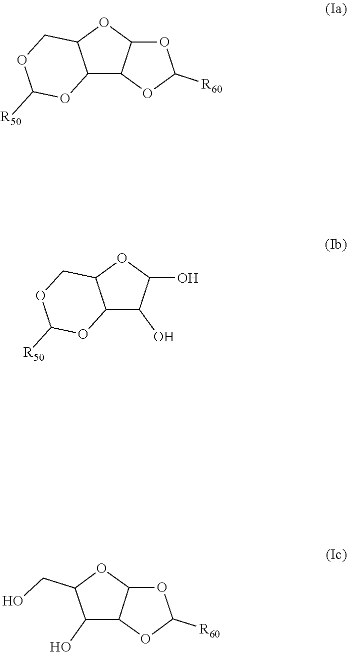
R₅₀ and R₆₀ are different form each other and are selected from the group consisting of —R₇₀, —ZR₇₀, —Z—OH, —Z—NH₂, —Z—SH, —Z—OC(O)R₇₀, —OC(O)R₇₀, —COOH and its corresponding salts, —C(O)NH₂, —C(O)NH—R₇₀, —C(O)N—(R₇₀)₂, —COOR₇₀, —Z—COOH and its corresponding salts, —Z—C(O)NH—R₇₀, —Z—C(O)NH₂, —Z—C(O)N—(R₇₀)₂, —Z—COOR₇₀, —CH(COOH)₂ and its corresponding salts, —CH(COOR₇₀)₂, and —Z—SO₃—
wherein R₇₀ is selected from the group consisting of a linear or branched C₁ to C₂₀ alkyl, (C₁ to C₁₀)-alkyloxy-(C₁ to C₁₀)-alkyl, C₂ to C₁₅ alkenyl, C₆ to C₁₂ aryl, C₃ to C₁₀ cycloalkyl, cycloalkylalkyl and cycloalkylalkenyl,
wherein Z is a linear or branched C₁ to C₁₀ alkyl, linear or branched C₃ to C₁₀ cycloalkyl, a linear or branched C₆ to C₁₀ aryl or a (C₁ to C₁₀)-alkyloxy-(C₁ to C₁₀)-alkyl, cycloalkylalkyl and cycloalkylalkenyl.

2. The compound of the general formula (Ib) or (Ic) according to claim 1, wherein R₅₀ or R₆₀ is —R₇₀ and
wherein —R₇₀ is a linear C₇ to C₁₉ alkyl.

3. The compound of the general formula (Ib) according to claim 2, wherein R₅₀ is —R₇₀ and
wherein —R₇₀ is a linear C₇ to C₁₉ alkyl.

4. The compound of the general formula (Ic) according to claim 2, wherein R₆₀ is —R₇₀ and
wherein —R₇₀ is a linear C₇ to C₁₉ alkyl.

5. The compound of the general formula (Ia) according to claim 1, wherein one of R₅₀ or R₆₀ is —R₇₀ and the other is —COOH or its corresponding salt and
wherein —R₇₀ is a linear C₇ to C₁₉ alkyl.

6. The compound of the general formula (Ib) according to claim 1, wherein R₅₀ is —R₇₀ and
wherein —R₇₀ is a linear C₂ to C₁₅ alkenyl.

7. A compound of the general formula (V) 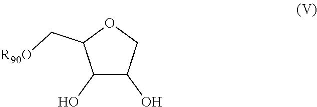
wherein R₉₀ is selected from the group consisting of a linear or branched C₁ to C₂₀ alkyl, (C₁ to C₁₀)-alkyloxy-(C₁ to C₁₀)-alkyl, C₂ to C₁₀ alkenyl, C₆ to C₁₂ aryl, C₃ to C₁₀ cycloalkyl, cycloalkylalkyl and cycloalkylalkenyl.

8. The compound of the general formula (V) according to claim 8, wherein R₉₀ is a linear C₇ to C₁₉ alkyl.

9. A cosmetic, food, frying, pharmaceutical or cleaning product comprising the compound according to claim 1.

10. A cosmetic, food, frying, pharmaceutical or cleaning product comprising the compound according to claim 7.
